(19) United States
(12) Patent Application Publication
(10) Pub. No.: US 2009/0285916 A1
(43) Pub. Date: Nov. 19, 2009

(54) METHODS OF DIAGNOSIS AND TREATMENT OF EQUINE LAMINITIS AND CUSHING'S SYNDROME

(75) Inventor: Susan Jane Alexia Haritou, Nottingham (GB)

Correspondence Address: Pabst Patent Group LLP, 1545 PEACHTREE STREET NE, SUITE 320, ATLANTA, GA, 30309, US

(73) Assignee: Pegasus Equine Diagnostics Limited

(21) Appl. No.: 12/299,166

(22) PCT Filed: May 2, 2007
(86) PCT No.: PCT/GB2007/001622

§ 371 Date: Jun. 4, 2009

(30) Foreign Application Priority Data
May 2, 2006 (GB) 0608647.4

(51) Int. Cl. A61K 36/38; C12Q 1/00; C12Q 1/54; A61K 31/4745; A61K 31/4045

(52) U.S. Cl. 424/730; 435/4; 435/14; 514/288; 514/415

(57) ABSTRACT

The invention provides a method for diagnosing a disorder or disease associated with an imbalance of monoamines in a subject, or of diagnosing susceptibility of the subject to the same, wherein the method comprising (a) providing a sample from a subject to be tested, (b) measuring the levels of dopamine, melatonin and/or serotonin in the sample; and (c) comparing the levels measured in step (b) with levels of dopamine, melatonin and/or serotonin associated with a reference population of subjects, wherein a difference in one or more of the levels measured in step (b) with the levels of dopamine, melatonin and serotonin associated with the reference population of subjects is indicative of a positive diagnosis. The invention further provides a method of prevention or treatment of a disorder or disease associated with an imbalance of monoamines, and kits for the same.

The present invention relates to methods for the prevention, diagnosis and treatment of disorders and diseases associated with an imbalance of monoamines (specifically dopamine, serotonin and melatonin). In particular, the invention provides methods for the prevention, diagnosis and treatment in horses of laminitis and equine Cushing's syndrome.

### BACKGROUND

Laminitis is a condition which is professionally acknowledged to be one of the most common causes of lameness and disability of horses and ponies in the UK. It may strike a horse or pony at any point during its life. From a technical standpoint the term laminitis most correctly implies that an inflammatory response is present in the submural lamnar structures of the hoof, but over time the term has become the designation for a specific disease. The scenarios hypothesised as being responsible for laminitis can be grouped into three types: dysfunction of the digital vasculature, the proposal of agents thought to directly affect the metabolic processes of the epidermal cells or basement membrane of the laminar epithelium, and the development of the condition subsequent to direct trauma to the hoof. Histologically, there is evidence of an inflammatory response present in the digital laminar tissues. Subsequently, other anatomical structures within the hoof become involved in a sequential cascade of pathological processes. For example, the bond between the dermal and epidermal laminae (the inter-laminar bond) is the only means of support of the distal phalanx within the hoof. If sufficient inter-laminar bonds are destroyed the animal may become foundered, i.e. the pedal bone may move distally within the hoof. These changes can occur within hours, to varying degrees, and tend to be irreversible.

Equine Cushing's syndrome may also occur in horses at any stage from about the age of seven years onwards (horses typically live up to a maximum of thirty five years). Horses suffering from equine Cushing's syndrome are particularly susceptible to laminitis.

Both laminitis and equine Cushing's syndrome may be life threatening, the mechanisms causing them are hotly debated by professionals, and the symptoms and effects of treatments may differ from horse to horse. Thus, throughout veterinary practices dealing with equines, there is a distinct need for an understanding of the underlying causes of these conditions and an effective method of prevention and treatment. Veterinarians report that the numbers of cases of both conditions is on the increase and that younger horses are being diagnosed. In an acute episode of laminitis, the worst-case scenario may mean that it is kindest to euthanise a horse after just a few days. The same may be said of Cushing's syndrome, due to the prevalence of laminitis associated with it.

Many therapeutic regimes have been described to treat laminitis but few published reports support their efficacy. Goetz & Comstock (1985) Proc. 31st Ann. Conv. Am. As. Equine Pract. p. 605-616 reported that in a group of 22 horses affected with varying severity and chronicity of laminitis 46% became sound following the use of adjustable heart bar shoes as part of the treatment regime, 27% were improved by at least 2 Obel grades and 27% required euthanasia. Allen et al. (1986) J. Am. Vet. Med. As. 189, 1604-1606 reported on the treatment of 13 cases of chronic refractory laminitis cases by the use of deep digital flexor tenotomy. Eleven cases improved, but did not become sound, and two were subjected to euthanasia. Baxter (1986) J. Am. Vet Med. Ass. 189, 326-329 recorded the results of treatment of 12 cases of distal displacement of the distal phalanx, three cases survived but remained lame. Hunt et al. (1991) Vet. Surg. 20, 15-20 used deep digital flexor tenotomy on 20 horses suffering from acute and chronic laminitis. Six cases survived more than six months following surgery, three of these remained lame and no case returned to athletic performance.

Little has been achieved in recent years in terms of improving the accuracy of the prognosis for laminitis cases. Colles & Jeffcott (1977) Vet. Rec. 100, 262-264 related the prognosis for a case of laminitis to the severity of onset, number of feet affected and speed of recovery although no results were clinical data was published. Stick et al (1982) J. Am. Vet. Med. Ass. 180, 251-253 produced a retrospective survey of referred laminitis cases. Their study indicated that horses with distal phalangeal rotation (relative to the dorsal hoof wall) of more than 11.5 degrees tended to remain lame. Stashak (1987) Lameness. Adams' Lameness in Horses. 4th Ed. T. S. Stashak, Lea and Febiger, Philadelphia. p. 498 suggests that only in exceptional circumstances should treatment be considered for cases in which the distal phalanx has penetrated the sole (solar prolapse). Eustace & Caldwell (1989) Equine Vet. J. 21, 370-373 demonstrated that animals with degrees of distal phalangeal rotation greater than the suggested threshold of 11.5 degrees can return to full athletic function if they are treated using a heart bar shoe and the technique of dorsal wall resection. Linford (1987) Ph. D Thesis, University of California. USA described the radiological measurement of wall thickness; an increase in this distance was found to be positively associated with laminitis and marginal fractures of the distal phalanx. Hunt (1993) Equine Vet. J. 25, 61-65. considered that the greater the severity of lameness the worse the prognosis for cases of laminitis.

Hence, there exists a need for new methods for the prevention, diagnosis and treatment of laminitis and equine Cushing's syndrome.

### SUMMARY OF THE INVENTION

According to a first aspect of the present invention, there is provided a method for diagnosing a disorder or disease associated with an imbalance of monoamines in a subject, or of diagnosing susceptibility of a subject to the same, wherein the method comprises the following steps:
(a) providing a sample from a subject to be tested;
(b) measuring the levels of dopamine, melatonin and/or serotonin in the sample; and
(c) comparing the levels measured in step (b) with levels of dopamine, melatonin and/or serotonin in a reference population of subjects
   wherein a difference in one or more of the levels measured in step (b) with the levels of dopamine, melatonin and serotonin associated with the reference population of subjects is indicative of a positive diagnosis.

In one embodiment, step (a) is omitted from the method of the invention (i.e. the step of providing the sample is not within the scope of the method as defined herein).

It will be appreciated by persons skilled in the art that the method of the invention is suitable for use on any subject which may experience an imbalance of monoamines. In particular, the method of the invention may be used on mammalian subjects, including humans, horses and dogs.

In a preferred embodiment, however, the subject to be tested is equine. For example, the subject may be a horse or pony.

By “monoamine” we specifically include the biogenic amines serotonin, dopamine and melatonin.

By “a disorder or disease associated with an imbalance of monoamines” in a subject we specifically include conditions associated with normal or abnormal aging processes, such as pineal degradation, metabolic syndrome (Reaven's syndrome), Cushing's syndrome (in mammals such as humans, horses and dogs, etc.) and laminitis (in horses and ponies).

In a preferred embodiment, the disorder or disease associated with an imbalance of monoamines is a disorder, disease or condition associated with aging.

Preferably, the disorder or disease associated with an imbalance of monoamines is lamnitis, equine Cushing's syndrome or equine metabolic syndrome (Reaven's syndrome).

Most preferably, the disorder or disease associated with an imbalance of monoamines is laminitis.

Although not wishing to be bound by theory, it is proposed that there is an identifiable systemic cause linking equine laminitis, equine Cushing's syndrome or equine metabolic syndrome; a progressive alteration in the relative ratios of the indole amines (serotonin and melatonin) and the catecholamine, dopamine, which increases the susceptibility of an individual to ‘disease’ during the course of normal development and ‘aging’.

For example, it is proposed that independently arising laminitis is caused by an acute, but transient biogenic amine imbalance. This links to the laminitis seen as a symptom of Cushing's syndrome in that this latter laminitis is believed to be a cumulative progression to the same state of imbalance, but on a chronic basis.

The neurotransmitter imbalance is thought to progress slowly in relation to cumulative ‘biological aging’ or it may arise as an acute, transient event instigated by one of the well-known ‘triggers’ of lamnitis. A transient incidence of ‘disease’, not associated with aging, may be understood if the appearance of the ‘disease’ is considered as dependent upon the following ‘susceptibility factors’:
1. An individual's genetics;
2. External influences affecting that individual; and/or
3. The process of ‘biological aging’

The present invention primarily relates to the third factor, in categorising laminitis, metabolic syndrome, and Cushing's syndrome as equine ‘diseases of aging’ and the clinical signs of progressive pineal degradation. However, it also directly relates to the second factor in describing the cause of independently arising laminitis.

In human medicine, changes or imbalances within the endocrine system are now being accepted as contributory to age-related conditions and ‘diseases’. One such condition is Metabolic Syndrome X (Reaven's syndrome) (see Reaven (1988) Diabetes 37: 1595-1607; Reaven et a (2001) “Syndrome X, The Silent Killer: The New Heart Disease Risk”, New York, Simon and Schuster) also known as Abdominal Obesity Metabolic Syndrome (see Bjorntorp (1991) Diabetes Care 14: 1132-1143), in which a number of signs and symptoms are observed due to insulin resistance (see Krentz (2002) “Insulin Resistance”, Oxford, Blackwell Science Ltd) and resulting hyperinsulinaemia. There is a suggestion that this may be prodromal to Cushing's syndrome (see Balasubramanyam (2002). “Is the ‘metabolic syndrome’ a mild form of cushing's syndrome? The curious story of 11beta-hydroxysteroid dehydrogenase”. Medscape Conference Coverage of ENDO 2002: The 84th Annual Meeting of The Endocrine Society, San Francisco, 19-22 Jun. 2002) and it has been proposed that a similar condition may also found in horses (see Johnson (2002) Vet. Clin. Equine 18: 271-293; Johnson et al. (2002) Vet. Clin. Equine 18: 219-236). However, what does not appear to have been considered previously is the possible link to these ‘diseases’ from events higher in a chain of cascade of metabolic control, i.e. to neuroendocrine considerations.

This possibility has been investigated via the Ontogenetic (or Neuroendocrine) Theory of Aging (see Dilman & Young (1994) “Development, Aging and Disease—A New Rationale for an Intervention Strategy”, Switzerland, Harwood Academic Publishers), which provides a framework for studying the growth, development, and subsequent ‘aging’ of an individual. The theory states that a loss of central and peripheral neurotransmitter and hormone sensitivity occurs gradually, over time, and causes a progressive shift in homeostasis throughout an individual's life. The resulting hormonal and metabolic shifts are thought to be causal in ‘aging’ and the ‘diseases of aging’ (see Dilman & Young (1994) “Development, Aging and Disease—A New Rationale for an Intervention Strategy”, Switzerland, Harwood Academic Publishers; Rozenewaig et al. (1987) Med. Hypothesis (England) 23(4):337-352; Rozencwaig & Walji (1997) “The Melatonin and Aging Sourcebook”, Prescott, Hohm Press; Simpkins et al. (1977) Endocrinology 100(6); 1672-1678). It has been proposed that the root of these effects is slow degradation and/or atrophy of the pineal gland (see Humbert & Pevet (1994) Ann. NY Acad. Sci. 719: 43-63) and progressive cumulative decrease in the indole amines it produces: serotonin and melatonin (see Dilman & Young (1994) “Development, Aging and Disease—A New Rationale for an Intervention Strategy”, Switzerland, Harwood Academic Publishers; Rozencwaig & Walji (1997) “The Melatonin and Aging Sourcebook”, Prescott, Hohm Press; Simpkins et al. (1977) Endocrinology 100(6): 1672-1678; Cano et al. (2003) Biological Rhythm Research 34(3): 279-294). However, there is another very important relationship which changes during aging: if a graph of serotonin and dopamine decline is plotted against years (see Dilman & Young (1994) “Development, Aging and Disease—A New Rationale for an Intervention Strategy”, Switzerland, Harwood Academic Publishers), the rate of decrease of the catecholamine, dopamine, is found to be much greater than that of serotonin. The pineal gland is thought to be implicated in this situation through the suggestion that melatonin sensitises and entrains dopamine synthesis/release rhythm (see Nava (2001) Cellular and Molecular Neurobiology 21(6): 605-616; Santanavanich et al. (2003) J. Pineal Res. 35(3): 169-176; Abilio et al. (2003) Life Sci. 72(26): 3003-3015; Curlewis (1992) Reprod Fertil Dev 4(1): 1-23).

However, in humans, as with horses, it is often found that these so-called ‘diseases of aging’ (for example Diabetes Mellitus—Type Two (DM) in humans, or laminitis in horses) may occur at any age, even though there may be a greater prevalence in older individuals. When all parts of an individual are considered as having not only a chronological age, but also a biological age, this discrepancy is more easily understood (see Dilman & Young (1994) “Development, Aging and Disease—A New Rationale for an Intervention Strategy”, Switzerland, Harwood Academic Publishers). ‘Biological age’ is described here as the state of a specific part of the body compared to its expected condition in terms of normal chronological years. For example, a fifteen-year-old human may show clinical signs of DM2, or a five-year-old horse may contract laminitis, due to obesity. It is not the state of excess adipose tissue per se that causes the clinical signs, but physiological changes and a systemic change in neuroendocrinology, which may mimic, however transiently, the internal environment found in a chronologically ‘older’ individual. Prior to manifestations of clinical signs, the human might be considered to have a pancreas in the same condition as a ‘healthy’, but older human, and the horse might be considered to have hoof physiology nearer to that of an older horse. Thus, the pancreas and the hoof may be observed to be of a greater biological age, than the chronological age of the individual.

It is proposed that development of a method for very early identification of the onset of signs of ‘biological aging’, as provided by the present invention, allows pre-emption of a laminitic episode in a susceptible equine individual, which may prove critical to a horse's future soundness and health. Additionally, rectifying an acute, transient state of imbalance thought to be instigated by any of the well-known ‘triggers’ of laminitis should result in the cessation of a laminitic episode. Similarly, rectifying the chronic state of imbalance suspected in Cushing's syndrome should result in cessation of the ‘Cushingoid’ state.

Two theories receiving interest in this area are the examination of fructans as a source of ‘grass founder’ (see Pollitt (2003) Hoofcare and Lameness Journal 76: 20-22; Pollitt (2001) “Equine Laminitis”, Kingston, Rural Industries Research and Development Corporation) and the description of Equine Metabolic Syndrome (see Johnson (2002) Vet. Clin. Equine 18: 271-293; Johnson et al. (2002) Vet. Clin. Equine 18: 219-236), a condition thought to increase susceptibility to laminitis (the theories are not mutually exclusive, however [see Pass (1998) Equine Vet. A Suppl. (26); 133-138]). The connection here is that it is thought that the link between events following ‘excess’ fructan ingestion and the subsequent clinical signs of laminitis to be a transient disruption of the ratios between the indoleamines, serotonin and melatonin, and the catecholamine, dopamine. Additionally, a state of chronic neurotransmitter imbalance is also believed to be the precursor to the clinical signs observed in Equine Metabolic Syndrome, which may, eventually, progress to Equine Cushing's Syndrome (see Van der Kolk et al. (2001) Equine Vet. J. 33(1): 110-112; Love (1993) Br. Vet. J. 149(2). 139-153).

In summary, the progressive degeneration of the pineal gland is thought to cause a change in relative ratios of serotonin, melatonin and dopamine. This results in the manifestations of ‘biological aging’, one of the three ‘susceptibility factors’ in defining how susceptible a particular individual will be to ‘disease’ (the other two are an individual's genetics and the external influences affecting that individual). Also, a transient ‘aged’ state may develop at any point in time, dependent upon the ‘susceptibility factors’, i.e. primarily genetics, or external influences affecting the individual, resulting in the same pathologies. For example, there are shared endocrinopathies in horses with ‘Pituitary-Dependent’ Cushing's syndrome (see Schott (2002) Vet. Clin. Equine 18; 237-270; Love (1993) Br. Vet. J. 149(2): 139-153), and cases of acute carbohydrate-overload laminitis (see Coffman & Colles (1983) Can. J. Comp. Med. 47(3): 347-351; Jeffcott & Field (1985) Vet. Rec. 116(17): 461-466; Field & Jeffcott (1989) Med. Hypotheses 30(3): 203210); a combination of the following may be in attendance in both scenarios: hypercorticism, hyperinsulinaemia, hyperglycaemia, glucose intolerance and hypertension.

The first step in the diagnostic methods of the present invention, Step (a), comprises providing a sample from a subject to be tested. The sample may be any sample which contains the monoamines to be measured, i.e. dopamine, melatonin and serotonin. For example, suitable samples include blood, serum, cerebrospinal fluid, saliva and urine.

Preferably, the sample is a blood plasma sample (which may be obtained by collecting blood in an EDTA-treated vessel and then centrifuging to collect the liquid plasma component). In certain circumstances, such as the measurement of serotonin, a blood serum sample may be used (which may be obtained by allowing the blood to clot, removing the clot and centrifuging to collect the liquid serum).

In one embodiment, a Prick Blood Test may be performed, using a device similar to that used by human diabetics or for performing the ‘heel test’ in babies.

Step (b) of the method of the invention comprises measuring the levels of dopamine, melatonin and serotonin in the sample to be tested. Methods suitable for measuring such monoamines are well known to persons skilled in art and commercial kits are available for making such measurements (for example, from Labor Diagnostika Nord GmbH, Nordhom, Germany).

In a preferred embodiment of the invention, the levels of dopamine, melatonin and/or serotonin are measured by radioimmunoassay (see Examples A to D, below). Radioimmunoassay involves mixing known quantities of radioactive antigen (frequently labelled with gamma-radioactive isotopes of iodine attached to tyrosine) with antibody to that antigen, then adding unlabeled or “cold” antigen and measuring the amount of labelled antigen displaced.

Initially, the radioactive antigen is bound to the antibodies. When “cold” (unlabeled, quest) antigen is added, the two compete for antibody binding sites—at higher concentrations of “cold” antigen, more of it binds to the antibody, displacing the radioactive variant. The bound antigens are separated from the unbound ones. The radioactivity of the bound antigens is measured and a binding curve is then plotted.

Alternatively, the levels of dopamine, melatonin and/or serotonin may be measured by reversed-phase high performance liquid chromatography (HPLC) or ELISA.

Preferably, step (b) comprises measuring the levels of all three monoamines, i.e. dopamine, melatonin and serotonin, in the sample. The relative ratios of these monoamines may also be calculated, for example the ratios of serotonin:melatonin, dopamine:melatonin and/or dopamine:melatonin.

It will be appreciated that the levels of dopamine, melatonin and/or serotonin may be measured directly or indirectly. Thus, metabolites of dopamine, melatonin and/or serotonin may be used to determine the levels of these monoamines in a sample. For example, 3-methoxytyramine (3MT) and/or dihydroxyphenylacetic acid (DOPAC) may be used as markers of dopamine (see Wynne et al., 2004, J. Chromatography B, 811:93-101).

Step (c) of the method of the invention comprises comparing the levels measured in step (b) with levels of dopamine, melatonin and/or serotonin associated with a reference population of subjects.

By “a reference population of subjects” we mean one or more control subjects, of the same species as the subject to be tested, which do not suffer from and/or are not susceptible to the disorder or disease associated with an imbalance of monoamines. Typically, the reference population of subjects is a population of healthy, adult subjects of the same species as the subject to be tested. The population may be a single reference or control animal, preferably however the population comprises a plurality of reference or control animals.

Thus, the method of the invention may comprise comparing dopamine, melatonin and/or serotonin levels in a subject to be tested with levels of those monoamines in healthy subjects matched for age, sex, weight, etc. Where the subjects are horses, the controls are preferably matched for one or more of breed/type, age, sex, height, weight and/or colour (horses of a certain colour may be of a certain genetic type). For example, the “a reference population of subjects” may be healthy, age-matched controls.

It will be appreciated by persons skilled in the art that levels of dopamine, melatonin and serotonin will fluctuate with the natural circadian rhythm of the subject. Hence, in a preferred embodiment the levels of dopamine, melatonin and serotonin associated with predetermined population of subjects are matched for time of sample collection. In other words, the sample is collected from the subject to be tested at the same time as samples were collected from the predetermined population of subjects.

In one embodiment, the samples are taken at about 1 pm, preferably in the Spring or Autumn.

Alternatively, samples may be collected at several time points on one or more days of testing.

Thus, in addition to comparisons in step (c) between absolute or relative amounts of dopamine, melatonin and/or serotonin levels between test and matched healthy animals, step (c) may also comprise looking for daily time shifts in release patterns and/or seasonal changes in release patterns.

Identification of an imbalance in relative amounts of dopamine, melatonin and/or serotonin may be indicative of a positive diagnosis (for example, of laminitis, equine Cushing's syndrome or equine metabolic syndrome, or susceptibility to developing the same).

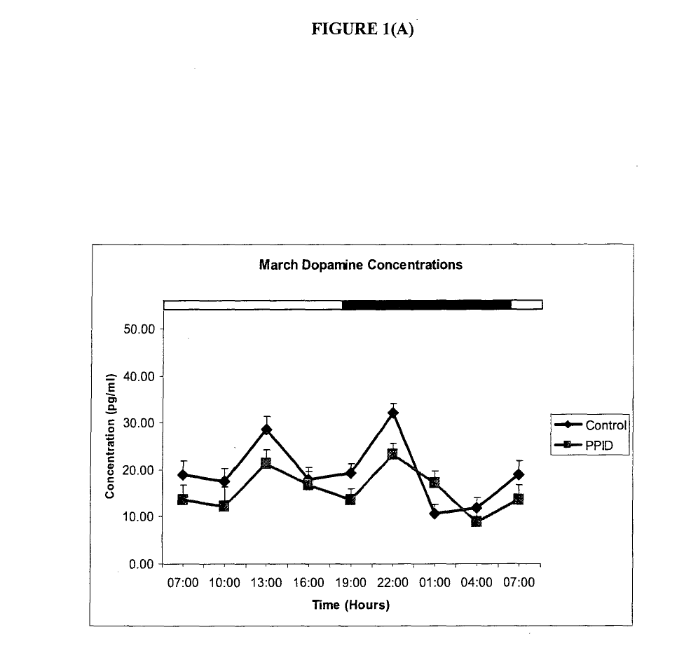

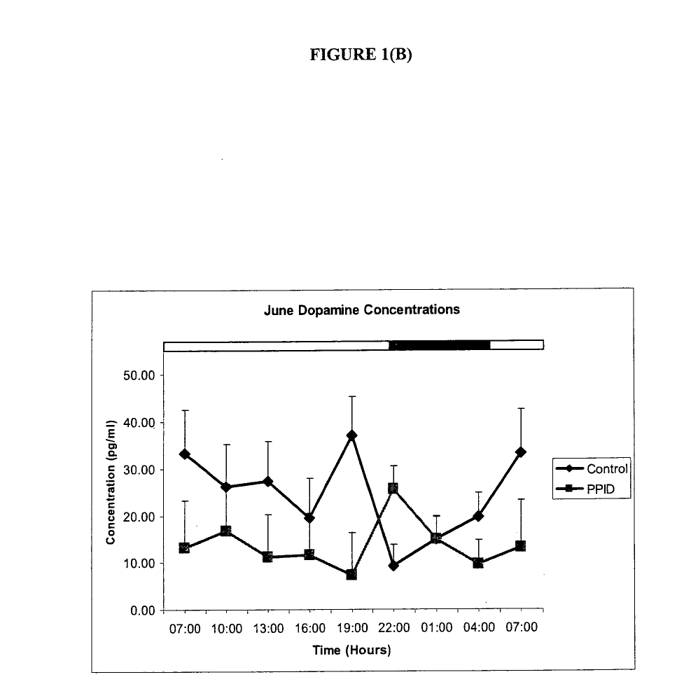

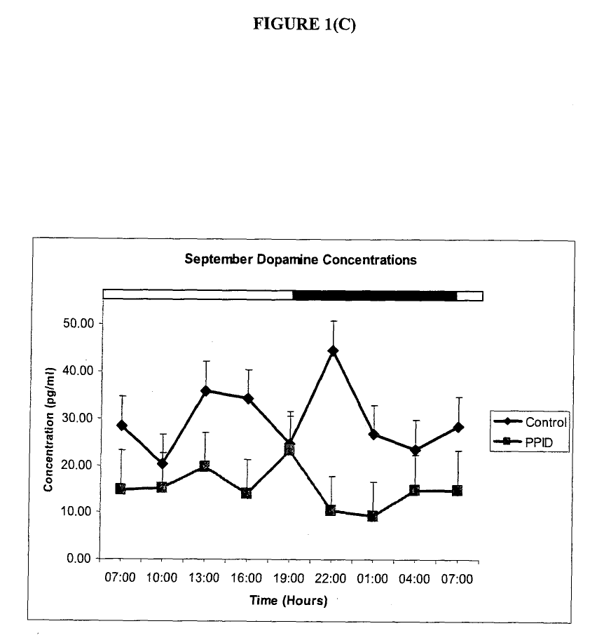

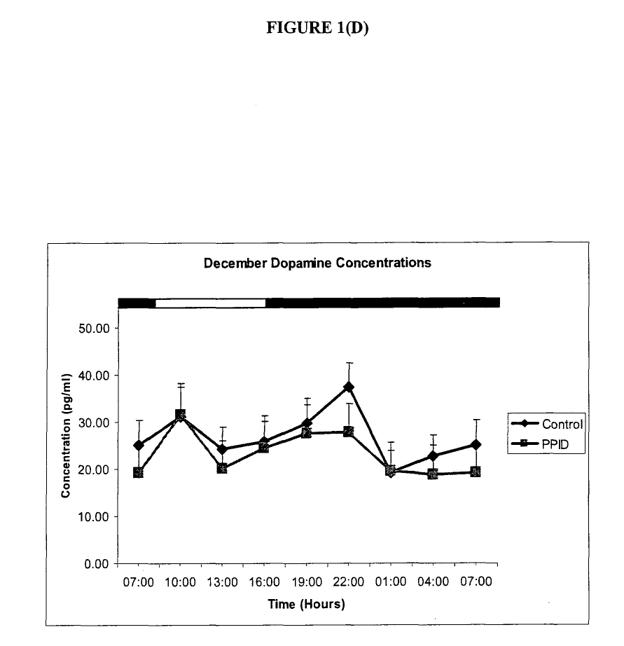

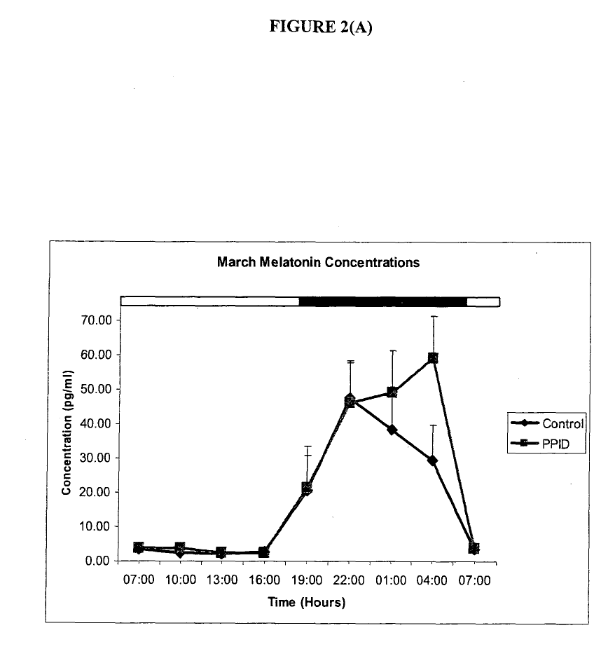

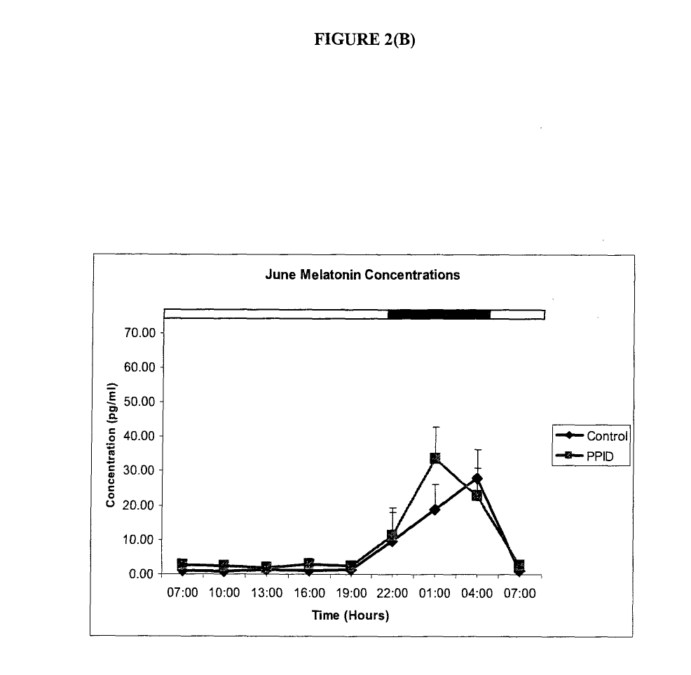

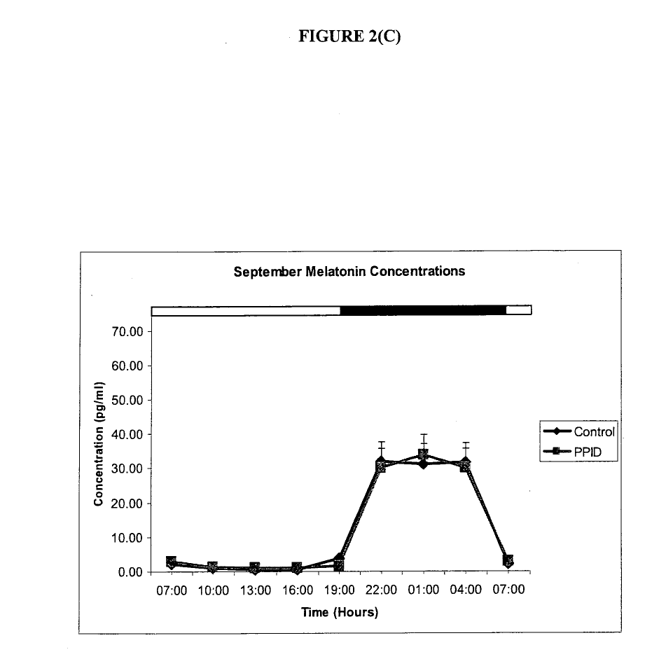

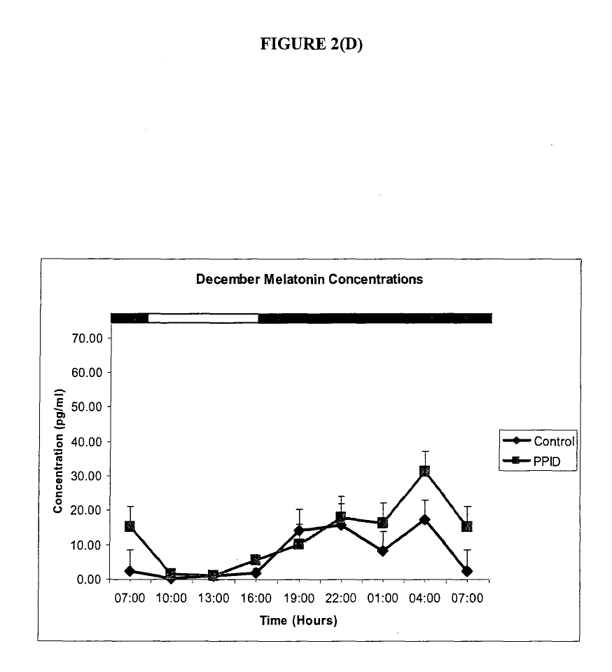

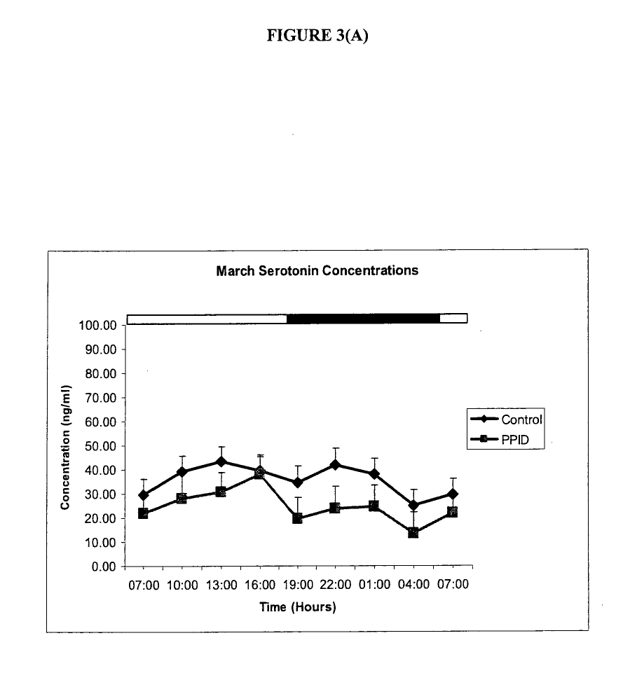

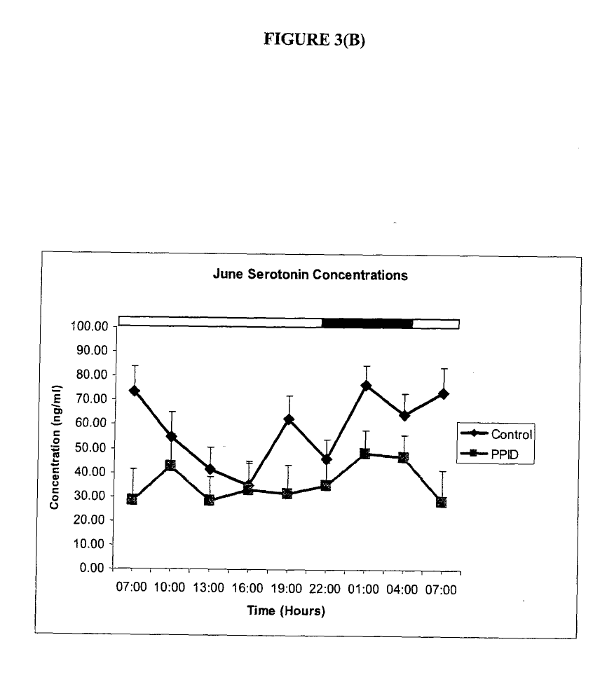

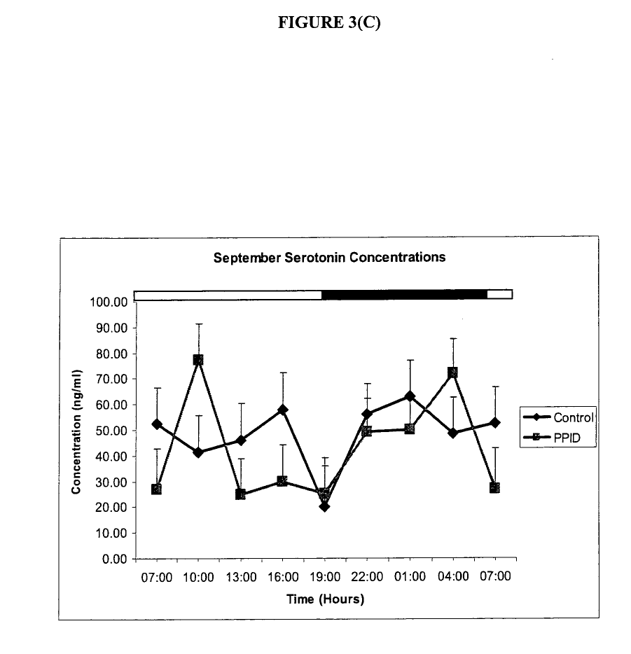

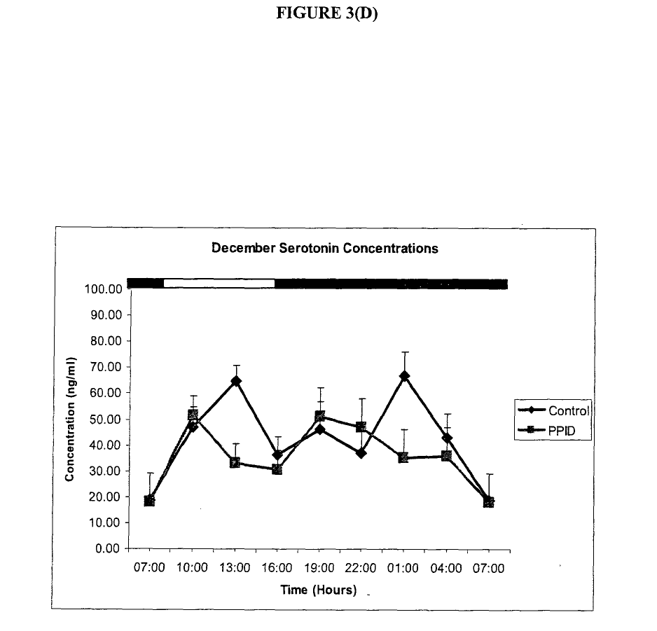

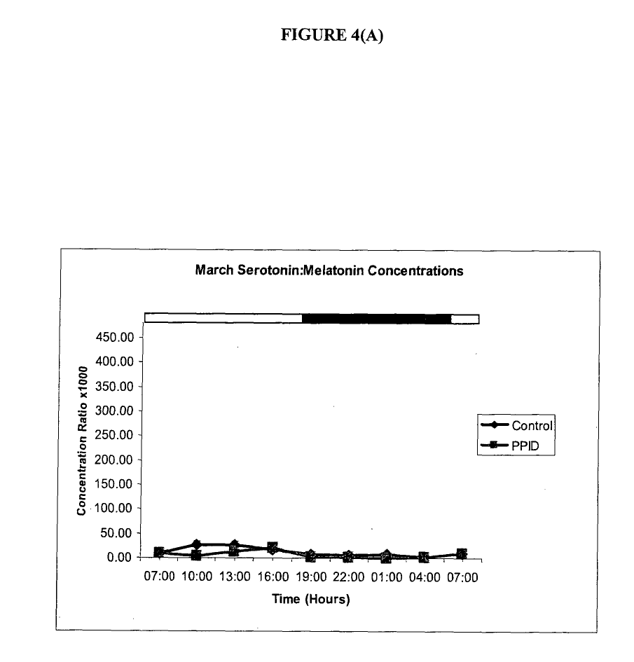

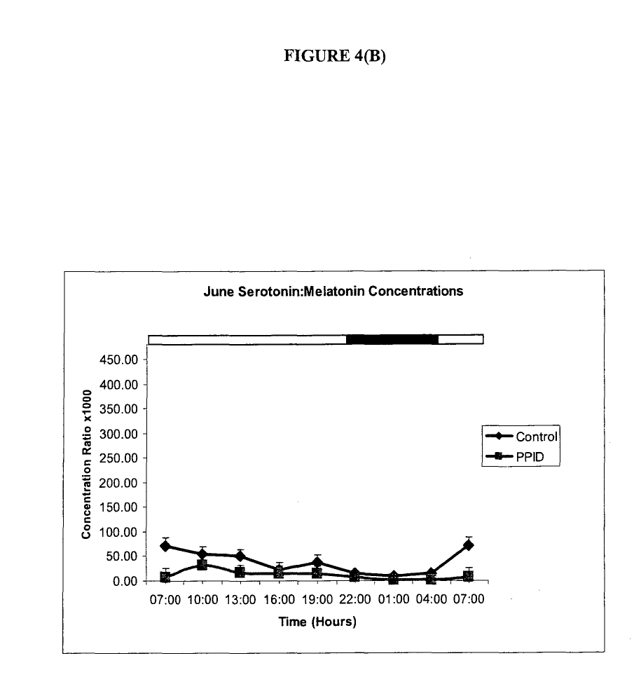

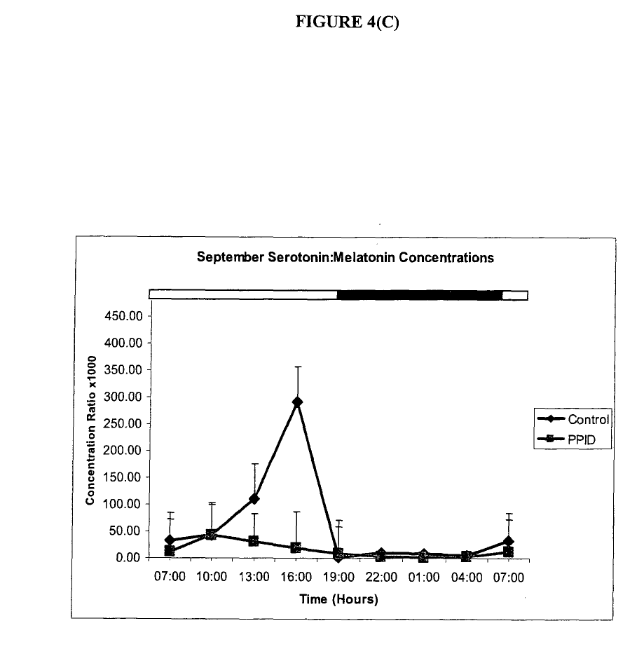

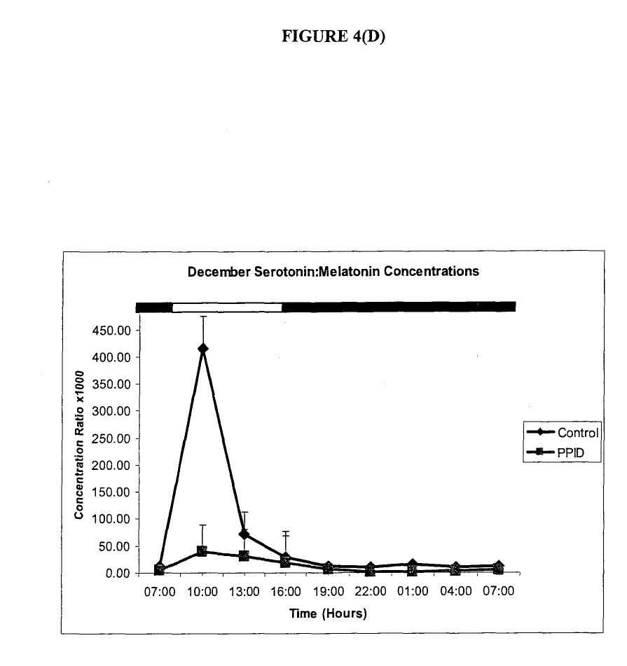

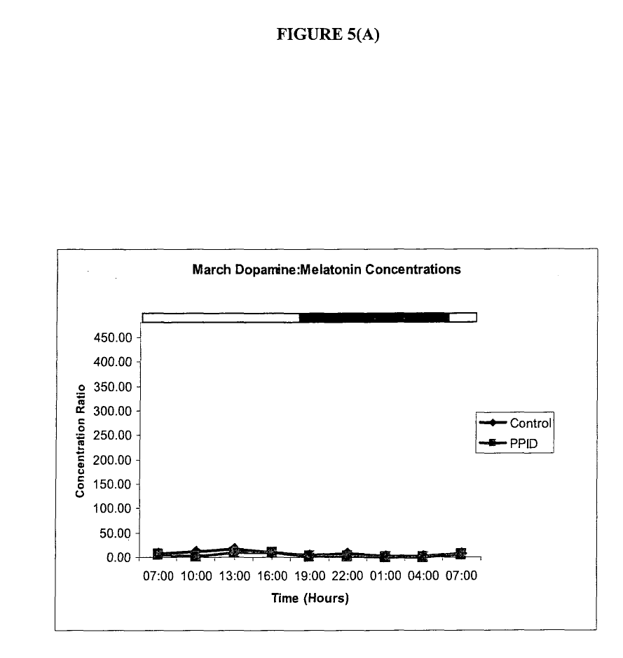

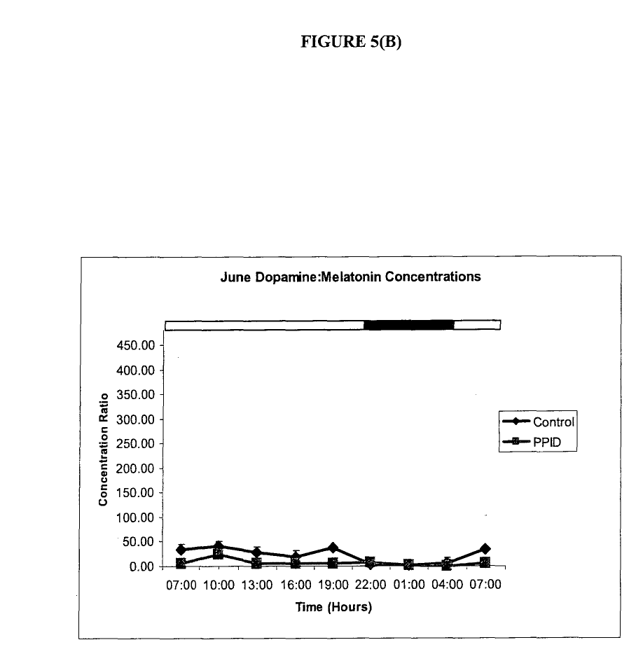

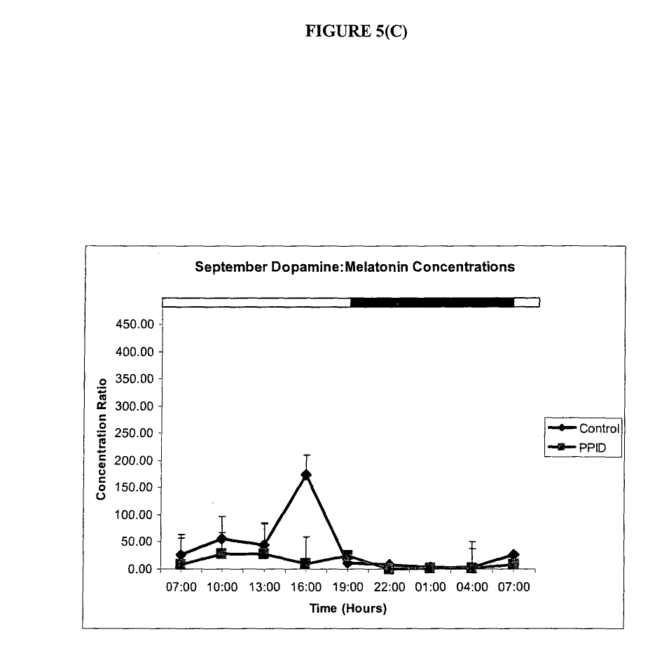

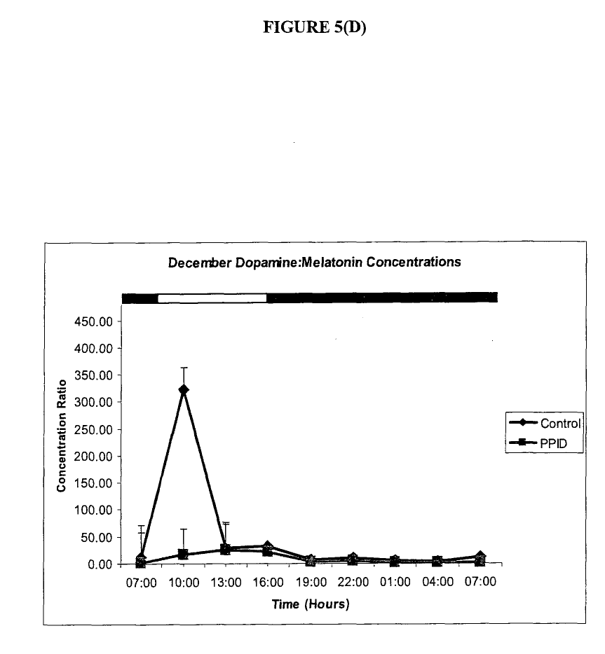

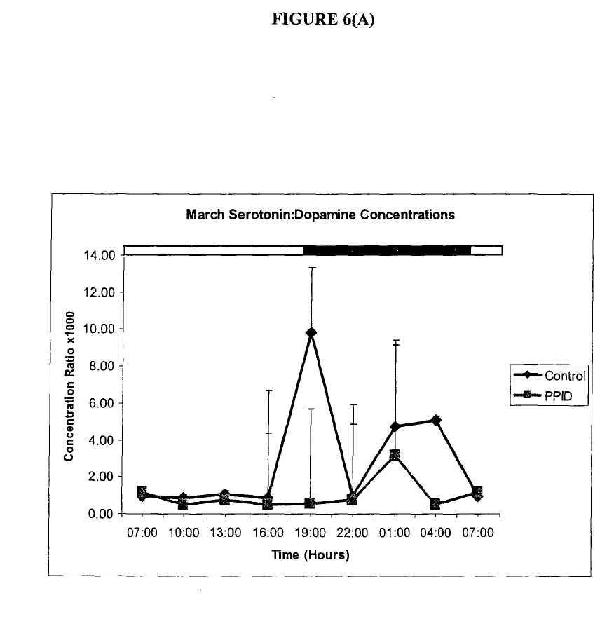

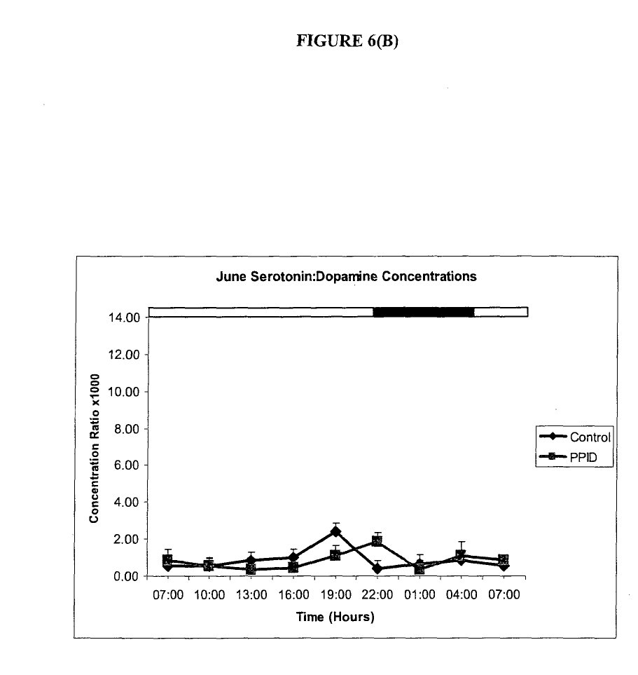

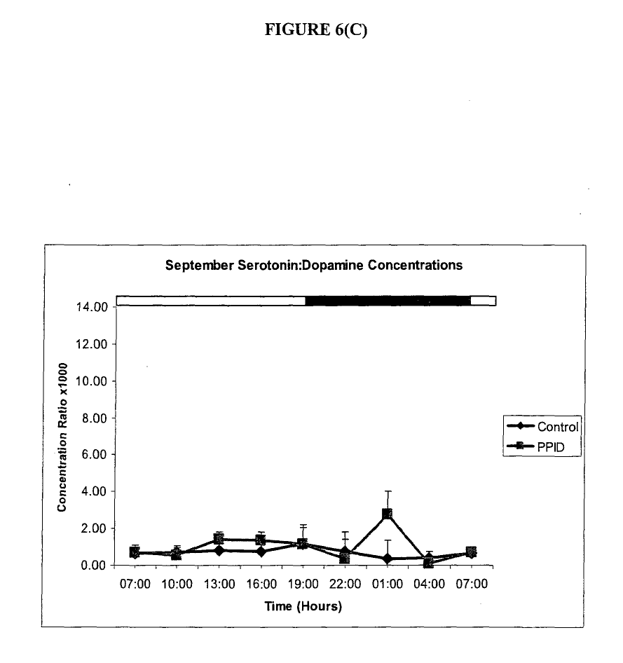

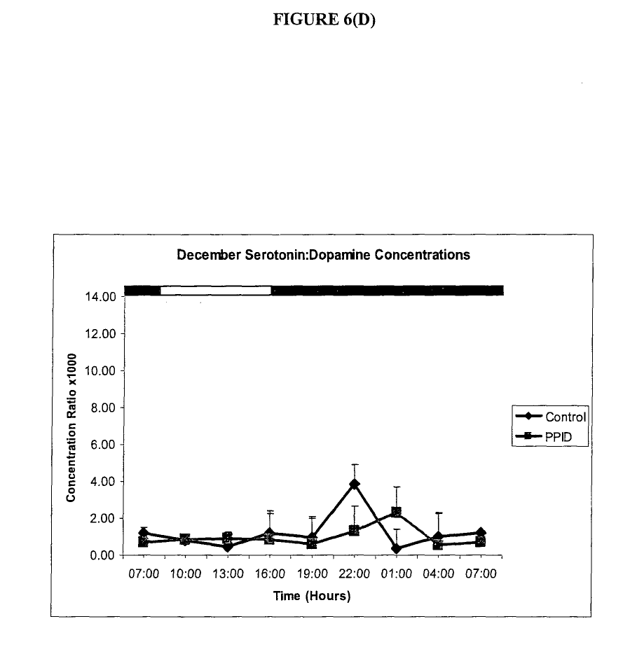

For example, the following determinants may be used, separately or in combination, to provide a positive diagnosis (e.g. of a horse having or susceptible to laminitis and/or equine Cushing's syndrome):
(a) A decrease in dopamine may be indicative of a positive diagnosis, most prevalent in the Surnmer months (e.g. May, June, July, August and/or September in the Northern hemisphere; see FIGS. 1b and 1c). This decrease may be associated with a reversal of the main acrophase (peak) in the circadian rhythm. For example, a shift in the evening peak of dopamine levels may be indicative of a positive diagnosis.
   Consequently, in one embodiment, dopamine levels are measured. Preferably such measurements are taken during the evening, for example between 4 pm and midnight, and preferably between or at 7 pm and 10 pm.
   Thus, blood plasma levels of dopamine taken between March and June, at or about 1 pm, of above about 30 pg/ml (for example above 28 pg/ml) may be indicative of a healthy horse, ie not suffering from laminitis and/or equine Cushing's disease.
(b) An increase in the duration of the night time melatonin peak, which is most pronounced in the Spring months (e.g. February, March and/or April in the Northern hemisphere; see FIG. 2a).
   Consequently, in one embodiment, melatonin levels are measured. Preferably such measurements are taken in the night time, for example between 10 pm and 7 am, and preferably between or at 1 am and 4 am.
(c) A decrease in levels of serotonin, which is most pronounced at night in the Summer months (e.g. May, June and/or July in the Northern hemisphere; see FIG. 3b). This decrease may also be evident in September at 1 pm and 4 pm, and in December at 1 am and/or 1 pm.
   Consequently, in one embodiment, serotonin levels are measured. Preferably such measurements are taken in the night time, for example between 10 pm and 6 am, and preferably between or at lam and 4 am.
   Thus, blood plasma levels of serotonin taken in September, at or about 1 pm or 4 pm, of below about 20 pg/ml (for example below 19 pg/ml) may be indicative of a horse suffering from laminitis and/or equine Cushing's disease. Similarly, blood plasma levels of serotonin taken in December, at or about 1 pm, of below about 30 pg/ml (for example below 31 pg/ml) may be indicative of a horse suffering from laminitis and/or equine Cushing's disease.
(d) A decrease in the serotonin:melatonin ratio, which is most prominent during daylight hours (see FIG. 4).
   Consequently, in one embodiment, serotonin and melatonin levels are measured. Preferably such measurements are taken during the daytime, for example between 7 am and 7 pm, and preferably between 7 am and 1 pm.
(e) A decrease in the dopamine:melatonin ratio, which is most prominent during daylight hours (see FIG. 5).
   Consequently, in one embodiment, dopamine and melatonin levels are measured. Preferably such measurements are taken during the daytime, for example between 7 am and 7 pm, and preferably between or at 7 am and 1 pm.
(f) A increase in the serotonin:dopamine ratio, which is most prominent at night, most prevalent in the Summer months (e.g. May, June, July, August and/or September in the Northern hemisphere; see FIGS. 6b and 6c).
   Consequently, in one embodiment, dopamine and serotonin levels are measured. Preferably such measurements are taken during the daytime, for example between 10 pm and 1 am.
   Alternatively, a decrease in the serotonin:dopamine ratio in June during the early evening (for example 7 pm) may be indicative of a positive diagnosis (see FIG. 6b).
(g) An increase in levels of serotonin in September at 4 am and/or 10 am (see FIG. 3c).
   Consequently, in a further embodiment, serotonin levels are measured. Preferably such measurements are taken in the morning, for example between 10 pm and 6 am, and preferably between or at 4 am and 10 am. Alternatively, measurements are taken in the morning about midday, for example at 1 pm.
   Thus, blood plasma levels of serotonin taken in September, at or about 1 am, of above about 80 pg/ml (for example above 77 pg/ml) may be indicative of a horse suffering from laminitis and/or equine Cushing's disease.

It will be appreciated by skilled persons that the months corresponding to particular seasons in the Northern hemisphere will differ in the Southern hemisphere.

Thus, in an exemplary embodiment, step (c) may comprise comparing the ratio of two or more of dopamine, melatonin and/or serotonin in test animals and matched, healthy animals. For example, a comparison may be made of the ratio of dopamine:serotonin, dopamine:melatonin, serotonin melatonin and/or dopamine:serotonin:melatonin.

However, in a particularly preferred embodiment, step (c) comprises comparing the dopamine:serotonin ratio in test animals and matched, healthy animals. An increase in this ratio may be indicative of a positive diagnosis (e.g. of equine Cushing's syndrome).

In a further preferred embodiment of the method of the invention, step (b) additionally comprises measuring one or more of glucose, adrenocorticotropic hormone (ACTH), cortisol and insulin and step (c) further comprises comparing the levels measured in step (b) with levels of glucose, ACTH, cortisol and/or insulin associated with a reference population of subjects.

Methods for the measurement of glucose, ACTH, cortisol and insulin in a sample are commercially available. For example, ELISA assays for the detection of insulin and cortisol are available from DRG International GmbH, Germany (Catalogue numbers EIA-2337 and EIA-1887, respectively). An ELISA-based assay for the detection of ACTH is available from Biomerica, Newport Beach, Calif. (Catalogue number 7023).

Measurement of the additional biochemical markers glucose, ACTH, cortisol and insulin may provide further diagnostic information, such as an indication of the organ(s) affected by the monoamine imbalance. Such further information may aid diagnosis. For example, high ACTH may be indicative of dopaminergic degradation and hence less dopamine (high ACTH may also lead to high cortisol levels). Likewise, high insulin may arise from a cascade effect of high serotonin or a serotonin:dopamine imbalance (specifically a higher serotonin level relative to that of dopamine, compared to normal state).

In another embodiment of the first aspect of the invention, the method is for diagnosing a disorder or disease associated with an imbalance of monoamines in a subject, or of diagnosing susceptibility of a subject to the same, prior to the subject exhibiting any clinical signs of the disorder or disease, i.e. early-stage diagnosis. For example, a vet may visit one of the ‘at risk’ horse types (such as native UK cob, fat, native UK pony, arab) which does not yet show any ‘clinical signs’, collect a blood sample and then perform the method of the invention to determine whether horse is susceptible to laminitis or developing Cushing's.

Of course, it will be appreciated that the method of the first aspect of the invention may also be used to provide a ‘confirmatory diagnosis’ following an external examination of a subject which indicated that the subject may be ill.

A second aspect of the invention provides a method for treating a subject suffering from a disorder or disease associated with an imbalance of monoamines, or from a susceptibility to the same, the method comprising the following steps:
(a) identifying an imbalance in one or more of the levels of dopamine, melatonin and/or serotonin in a sample from the subject compared to levels of dopamine, melatonin and/or serotonin in a corresponding sample from a reference subject using a method according to the first aspect of the invention; and
(b) administering one or more agent(s) in order to restore the (absolute and/or relative) levels of dopamine, melatonin and/or serotonin in the subject to levels of dopamine, melatonin and serotonin associated with reference subjects.

By ‘treatment’ we include both therapeutic and prophylactic treatment of the subject. Thus, the term ‘treating’ specifically encompasses the prevention and/or therapeutic treatment of a disorder or disease associated with an imbalance of monoamines.

It will be appreciated that the one or more agent(s) are administered in step (b) is an effective amount. By ‘effective amount’ we mean a concentration or amount of a compound which may be used to produce a favourable change in the disorder or disease being treated, whether that change is a remission, a favourable physiological result, a reversal or attenuation of the disorder or disease being treated, the prevention or the reduction in the likelihood of a disorder or disease state occurring, depending upon the disorder or disease treated. Where agents are used in combination, each of the agents may be used in an effective amount, wherein an effective amount may include a synergistic amount.

By “reference subjects” we mean one or more subjects which do not suffer from, and/or are not susceptible to, the disorder or disease associated with an imbalance of monoamines. Typically, the reference subjects are matched is healthy adults.

Preferably, the subject to be tested is equine.

Advantageously, the disorder or disease associated with an imbalance of monoamines is laminitis, equine Cushing's syndrome or equine metabolic syndrome (Reaven's syndrome).

The rationale underlying the treatment method of the second aspect of the invention is to restore the levels of dopamine, melatonin and/or serotonin in a subject suffering from a disorder or disease associated with an imbalance of monoamines, or from a susceptibility to the same, to levels of these monoamines associated with healthy subjects. It will be appreciated that it may not be necessary to restore the absolute levels of dopamine, melatonin and/or serotonin to those levels in healthy subjects. Rather, the aim is to restore the relative amounts of these monoamines, i.e. the balance of these monoamines, in the subject being treated.

Thus, the present invention provides a method of treating a subject suffering from, or susceptible to, a disorder or disease associated with an imbalance of monoamines, which method comprises administering one or more agent(s) so as to restore the relative levels of dopamine, melatonin and/or serotonin in the subject to the relative levels of dopamine, melatonin and/or serotonin associated with reference (healthy) subjects.

Any agent known to modulate the level of one or more of dopamine, melatonin and/or serotonin may be administered to the subject. Preferable, however, step (b) comprises administering one or more agents selected from the group consisting of dopamine agonists, dopamine antagonists, serotonin agonists, serotonin antagonists, melatonin agonists and melatonin antagonists.

In a preferred embodiment, wherein the subject is identified as having low is dopamine levels, the one or more agents administered in step (b) includes a dopamine agonist. By “dopamine agonist” we include any agent which increases dopamine levels or mimics the effect of the same, such as dopamine precursors, dopamine releasing agents, dopamine re-uptake blockers, direct dopamine agonists and dopamine autoreceptor antagonists.

Preferably, the dopamine agonist is selected from the group consisting of pergolide, cabergoline, ropinirole, bupropion, apomorphine, L-DOPA, dopamine, bromocriptine, lisuride, selegiline, St John's Wort, pramipexole, amantadine, chasteberry (Agnus castus), blueberries and other dark fruits.

Most preferably, the dopamine agonist is pergolide.

In a further preferred embodiment, the one or more agents administered in step (b) includes a serotonin antagonist. By “serotonin antagonist” we include any agent which reduces levels of serotonin or which mimics the effect of a reduction in serotonin levels, e.g. by blocking serotonin receptors. Thus, by serotonin antagonist we include direct-acting serotonin receptor antagonists as well as serotonin metabolism and/or release inhibitors.

Preferably, the serotonin antagonist is selected from the group consisting of feverfew (the active component of which is thought to be parthenolide), cyproheptadine, methysergide, isocarboxazid, phenelzine, selegiline and tranylcypromine.

Most preferably, the serotonin antagonist is feverfew or cyproheptadine.

Thus, step (b) may comprise administration of a combination of drugs to correct an imbalance in the dopamine and serotonin systems. For example, a combination of pergolide and a serotonin antagonist such as feverfew or cyproheptadine may be administered.

Optionally, the one or more agents administered in step (b) includes a melatonin agonist. By “melatonin agonist” we include any agent which increases melatonin levels or mimics the effect of the same, such as melatonin precursors, melatonin releasing agents, melatonin re-uptake blockers, direct melatonin agonists and melatonin autoreceptor antagonists.

Preferably, the melatonin agonist is melatonin.

In a particularly preferred embodiment of the second aspect of the invention, step (b) comprises administering a dopamine agonist, a serotonin antagonist and melatonin. The dopamine agonist, a serotonin antagonist and melatonin may be separate agents or may be one or two agents which provide these multiple pharmacological effects.

Advantageously, step (b) comprises administering pergolide, feverfew and melatonin.

Conveniently, in step (b) the one or more agents are administered at a time of day selected so as to mimic the normal circadian rhythm of dopamine, melatonin and serotonin.

A third aspect of the invention provides a diagnostic kit for performing a method according to the first aspect of the invention, the kit comprising:
(a) one or more reagent(s) for the detection of dopamine;
(b) one or more reagent(s) for the detection of serotonin; and/or
(c) one or more reagent(s) for the detection of melatonin.

Preferably, the kit comprises:
(a) one or more reagent(s) for the detection of dopamine by radioimmunoassay (e.g. an anti-dopamine antibody and radiolabelled dopamine);
(b) one or more reagent(s) for the detection of serotonin by radioimmunoassay (e.g. an anti-serotonin antibody and radiolabelled serotonin); and/or
(c) one or more reagent(s) for the detection of melatonin by radioimmunoassay (e.g. an anti-melatonin antibody and radiolabelled melatonin).

Advantageously, the diagnostic kit further comprises instructions for performing a method according to the first aspect of the invention.

The invention further provides the use of a kit according to the third aspect of the invention to measure levels of dopamine, serotonin and/or melatonin in a horse or pony.

A fourth aspect of the invention provides a treatment kit for use in a method according to the second aspect of the invention, the kit comprising:
(a) one or more agent(s) for modulating the level of dopamine in a subject (e.g. a horse);
(b) one or more agent(s) for modulating the level of serotonin in a subject; and/or
(c) one or more agent(s) for modulating the level of melatonin in a subject.

Preferably, the treatment kit comprises a dopamine agonist, a serotonin antagonist, and a melatonin agonist.

More preferably, the treatment kit comprises pergolide, feverfew and melatonin.

Advantageously, the diagnostic kit further comprises instructions for performing a method according to the second aspect of the invention.

A fifth aspect of the invention provides an animal feed, or a supplement therefor, comprising:
(a) one or more agent(s) for modulating the level of dopamine in an animal;
(b) one or more agent(s) for modulating the level of serotonin in an animal; and/or
(c) one or more agent(s) for modulating the level of melatonin in an animal.

Thus, in one embodiment, the feed comprises a combination of substances which are able to correct an imbalance in the dopamine and serotonin systems. For example, a combination of a dopamine agonist, precursor or agent mimicking dopamine, such as blueberry extract, and a serotonin antagonist or agent mimicking serotonin antagonism, such as feverfew, may be incorporated into the manufacture of the product.

Preferably, the animal feed or supplement comprises tyrosine and parthenolide.

In a further embodiment, the animal feed or supplement comprises precursors for dopamine, serotonin and/or melatonin.

In one embodiment, the animal feed or supplement is suitable for use in horses and/or ponies.

In a related aspect of the invention, there is provided a pharmaceutical composition for use in horses comprising a dopamine agonist, a serotonin antagonist, and a melatonin agonist. For example, the composition may comprise melatonin (optionally in controlled-release form; e.g. 1.5 mg), pergolide (such as the mesilate; e.g. 0.75 mg) and parthenolide (e.g. 3.2 mg). Such active agents may be formulated with known pharmaceutically acceptable excipients and/or carriers.

It will be appreciated by persons skilled in the art that the above animal feed, supplement and/or pharmaceutical composition may be formulated as a solid dosage form, such as spheroids/pellets, minitabs and granules.

Aspects and embodiments of the present invention will now be illustrated, by way of example. Further aspects and embodiments will be apparent to those skilled in the art.

FIG. 1: Circadian rhythm of PPID and control group blood plasma dopamine concentrations, by sampling period (from t-test pairwise comparisons of the differences in adjusted means at each time point, analysed by grouping the data by photophase and scotophase).

FIG. 2: Circadian rhythm of PPID and control group blood plasma melatonin concentrations, by sampling period (from t-test pairwise comparisons of the differences in adjusted means at each time point, analysed by grouping the data by photophase and scotophase).

FIG. 3: Circadian rhythm of PPID and control group blood plasma serotonin concentrations, by sampling period (from t-test pairwise comparisons of the differences in adjusted means at each time point, analysed by grouping the data by photophase and scotophase).

FIG. 4: Circadian rhythm of PPID and control group blood plasma Serotonin:Melatonin ratio, by sampling period (from t-test pairwise comparisons of the differences in adjusted means at each time point, analysed by grouping the data by photophase and scotophase).

FIG. 5: Circadian rhythm of PPID and control group blood plasma Dopamine:Melatonin ratio, by sampling period (from t-test pairwise comparisons of the differences in adjusted means at each time point, analysed by grouping the data by photophase and scotophase).

FIG. 6: Circadian rhythm of PPI and control group blood plasma Dopamine:Serotonin ratio, by sampling period (from t-test pairwise comparisons of the differences in adjusted means at each time point, analysed by grouping the data by photophase and scotophase).

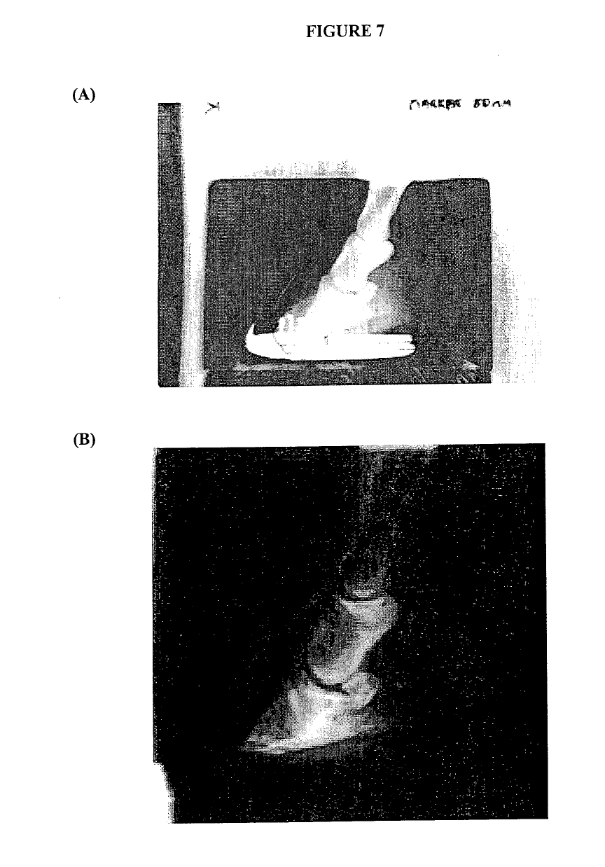

FIG. 7: shows exemplary X-rays of the hoof of a horse suffering from laminitis (a) before and (b) after treatment according to the present invention.

### EXAMPLES

### Example A

### Radioimmunoassay for the Quantitative Determination of Dopamine I

A quantitative determination of dopamine levels in a sample may be performed by radioimmunoassay using commercially available kits, such as the ‘Dopamine RIA’ kit of Labor Diagnostika Nord GmbH & Co KG, Nordhorn, Germany (Catalogue No. BA-0300).

### Reagents and Equipment

### Reagents for the Extraction and Acylation

Standard A, 0 ng/ml (Dopamine)
Standard C, 22.5 ng/ml (Dopamine)
Standard C, 75 ng/ml (Dopamine)
Standard D, 225 ng/ml Dopamine)
Standard E, 750 ng/ml (Dopamine)
Standard F, 2,250 ng/ml (Dopamine)

### Control 1

### Control 2

### Acylation Buffer

### Acylation Reagent

Assay Buffer, contains 1 M HCl

### Extraction Buffer

Macrotiter Plate, 48 wells, coated with boronate affinity gel
Hydrochloric Acid, contains 0.025 M HCl

### Reagents for the Radioimmunoassay

### Coenzyme, S-adenosyl-L-methionine

Enzyme, lyophilized, contains the enzyme catechol-O-methyltransferase

### Enzyme Buffer

Precipitating Reagent, goat anti-rabbit serum in PEG phosphate buffer.

Dopamine Antiserum, from rabbit ¹²⁵I-Dopamine, activity <100 kBq

### Reagents of the Additional Cat RIA Extraction Set

### Acylation Reagent

Assay Buffer, contains 1 M HCl

### Extraction Buffer

Macrotiter Plate, 48 wells, coated with boronate affinity gel

### Additional Materials and Equipment Required

Automatic pipettes for 10, 25, 50, 100, 150, 250 and 1,000 μl
Polystyrene tubes and suitable rack
Temperature controlled water bath or heating block (37° C.)
Centrifuge capable of at least 3,000×g
Suitable device for aspirating or decanting
Vortex mixer
Orbital shaker (capable of shaking between 400-900 rpm-ref. to an orbital shaker with a shaking orbit of 5 mm)
Gamma counter
Distilled water

### Sample Collection and Storage

### Plasma

EDTA plasma samples are required for the assay. Physical and psychical stress usually causes a high increase of the catecholamine concentration. Therefore, it is recommended to let the subject rest for 20 to 30 minutes after the venipuncture and before collecting the blood sample. Haemolytic and especially lipemic samples should not be used for the assay, because false low values will be obtained with such samples. The plasma samples can be stored up to 6 hours at 2-8° C. For a longer period (up to 6 months) the samples should be stored at −20° C.

### Urine

It is possible to use spontaneous as well as 24-hours urine. The total volume of urine excreted during a 24-hours period should be collected and mixed in a single bottle containing 10-15 ml of 6 M hydrochloric acid as preservative. Avoid exposure to direct sunlight. Determine the total volume and take an aliquot for the measurement. Urine samples can be stored at −20° C. for at least 6 months.

### Assay Procedure (for Determination of Dopamine in Plasma)

### Preparation of Standards and Controls

The standards and controls must be diluted 1+9 with dist. H₂O.

### Sample Preparation, Extraction and Acylation

Allow reagents and samples—with the exception of Precipitating Reagent—to reach room temperature. Each 10 μl of standards and controls are extracted. Each 600 μl of plasma samples are extracted. Determinations in duplicates are recommended.
1. Pipette 10 μl of diluted Standard A—F and 10 PI of diluted Control 1 & 2+Add 500 μl of distilled water to these wells to correct for volume. Pipette 600 μl of plasma sample into the respective wells for extracting dopamine.
2. Pipette 50 μl of Assay Buffer into all wells
3. Pipette 50 μl of Extraction Buffer into all wells.
4. Cover the plate with adhesive foil and incubate 60 min. at room temperature on an orbital shaker (600-900 r/min).
5. Remove the foil and discard. Immediately decant the plate and remove residual liquid by tapping the inverted plate on a paper towel.
6. Pipette 150 μl of Acylation Buffer into all wells.
7. Pipette 25 μl of Acylation Reagent into all wells.
8. Incubate the plate without foil for 15 minutes at room temperature on an orbital shaker (600-900 r/min).
9. Decant the plate immediately and remove residual liquid (see 5).
10. Pipette 1 ml of distilled water into all wells.
11. Incubate the plate without foil for 5 minutes at room temperature on an orbital shaker (600-900 r/min).
12. Decant the plate immediately and remove residual liquid (see 5).
13. Pipette 100 μl of Hydrochloric Acid (0.025 M) into all wells to elute the dopamine.
14. Cover the plate with adhesive foil and incubate for 10 minutes at room temperature on an orbital shaker (600-900 r/min). Caution: Do not decant the supernatant thereafter!

### Dopamine RIA

1. Pipette 90 μl of the extracted standards, controls and plasma samples into the respective tubes.
2. Pipette 25 μl of Enzyme Solution into all tubes (except totals).
3. Mix thoroughly and incubate for 30 minutes at 37° C.
4. Pipette 50 μl of the ¹²⁵I-Dopamine into all tubes.
5. Pipette 50 μl of Dopamine Antiserum into all tubes (except totals and NSB).
6. Mix thoroughly and centrifuge for 1 minute at 500×g
7. Incubate for 15-20 hours (overnight) at 2-8° C.
8. Mix the chilled (2-8° C.) Precipitating Reagent thoroughly, pipette each 1 ml into all tubes (except totals) and mix on a vortex.
9. Incubate for 15 minutes at 2-8° C.
10. Centrifuge for 15 minutes at 3,000×g, if possible in a refrigerated centrifuge.
11. Decant or aspirate the supernatant carefully (except totals). Beat out the tubes and leave it upside for 2 minutes.
12. Count all tubes for 1 minute in a gamma-counter.

### Calculation of Results

Concentration of the standards: Dopamine; A=0 ng/ml/B=22.5 ng/ml/C=75 ng/ml/D=225 ng/ml/E=750 ng/ml/f=2,250 ng/ml

Subtract the mean cpm of the non-specific binding NSB from the mean cpm of Zero Reference (=Standard A), Standards B-F, Control 1 & 2 and patient samples. Construct a standard curve by plotting the percentage of cpm of each standard in relation to the cpm of the Zero Reference ((B−NSB)/(B0−NSB) in %) versus its corresponding concentration. The concentration of the controls and patient samples can then be read off the standard curve by using their percentage of (B−NSB)/(B0−NSB).

Urine samples and controls: The concentrations of the urine samples and the Controls 1 & 2 can be read directly from the standard curve.

Plasma samples: The read concentrations of the plasma samples have to be divided by 600.

### Quantitative Determination

The calibration curve from which the concentration of dopamine in the samples can be taken, is obtained by plotting % B/B0 values measured for the 6 standards (linear, y-axis) against the corresponding concentrations (logarithmic, x axis). The results for unknowns can be calculated using one of the following curve-fitting techniques: spline fits, Akima or four-parameter logistic.

### Example B

### Radioimmunoassay for the Quantitative Determination of Dopamine II

A quantitative determination of dopamine levels in a sample may be performed by radioimnunoassay using commercially available kits, such as the ‘Dopamine Research RIA’ kit of Labor Diagnostika Nord GmbH & Co KG, Nordhorn, Germany (Catalogue No. BA-5300).

### Reagents and Equipment

### Reagents for the Extraction and Acylation

Standard A, 0 pg/μl (Dopamine)
Standard B, 9 μg/μl (Dopamine)
Standard C, 30 pg/μl (Dopamine)
Standard D, 90 pg/μl (Dopamine)
Standard E, 300 pg/μl (Dopamine)
Standard F, 900 pg/μl (Dopamine)

### Control 1

### Control 2

### Acylation Buffer

Acylation concentrate

### Acylation Diluent

Assay Buffer, contains 1 M HCl

### Extraction Buffer

Macrotiter Plate, 48 wells, coated with boronate affinity gel
Hydrochloric Acid, contains 0.025 M HCl

### Reagents for the Radioimmunoassay

### Coenzyme, S-adenosyl-L-methionine

Enzyme, lyophilized, contains the enzyme catechol-O-methyltransferase

### Enzyme Buffer

Precipitating Reagent, goat anti-rabbit serum in PEG phosphate buffer.
Dopamine Antiserum, from rabbit
¹²⁵I-Dopamine, activity <100 kBq,

### Additional Materials and Equipment Required but not Provided in the Kit

Automatic pipettes for 10, 25, 50, 100, 150, 250 and 1,000 μl
Polystyrene tubes and suitable rack
Temperature controlled water bath or heating block (37° C.)
Centrifuge capable of at least 3,000×g
Suitable device for aspirating or decanting
Vortex mixer
Orbital shaker (capable of shaking between 400-900 rpm-ref. to an orbital shaker with a shaking orbit of 5 mm)
Gamma counter
Distilled water

### Sample Collection and Storage

Tissue homogenates, dialysates, other ultra small sample volumes and plasma can be stored up to 6 hours at 2-8° C. For a longer period (up to 6 months) the samples should be stored at −20° C.

Preferred sample volume per measurement ˜500 μl.

### Assay Procedure (for Determination of Dopamine)

### Preparation of Acylation and Enzyme Solutions

The acylation solution is prepared freshly prior to the assay (not longer than 60 minutes in advance). The acylation concentrate is diluted 1+60 with the acylation diluent.

The enzyme solution is also prepared freshly prior to the assay (not longer than 10-15 minutes in advance). The enzyme is reconstituted with distilled water and mixed thoroughly, before addition of the co-enzyme and enzyme buffer.

### Sample Preparation

Tissue homogenates: Avoid chaotropic chemicals like perchloric acid. It is not necessary to deproteinate cytosols. Whenever possible, homogenise tissue sample in 0.1 M HCl.

Dialysates and urine: Store samples acidified (but avoid excess acid).

### Extraction and Acylation (Sample Volume 500 μl)

1. Pipette 10 μl of diluted Standard A—F, 10 μl of diluted Control 1 & 2 and 500 μl of samples into respective wells of extraction plate. Add 490 μl of distilled water to wells containing standard and control solutions.
2. Pipette 50 μl of Assay Buffer into all wells
3. Pipette 50 μl of Extraction Buffer into all wells.
4. Cover the plate with adhesive foil and incubate 60 min. at room temperature on an orbital shaker (600-900 r/min).
5. Remove the foil and discard. Immediately decant the plate and remove residual liquid by tapping the inverted plate on a paper towel.
6. Pipette 1 μl of distilled water into all wells.
7. Shake for 5 minutes at room temperature on an orbital shaker (600-900 r/min).
8. Immediately empty plate and eliminate residual fluid on a paper towel.
9. Pipette 150 μl of Acylation Buffer into all wells.
10. Pipette 25 μl of Acylation Reagent into all wells.
11. Incubate the plate for 15 minutes at room temperature on an orbital shaker (600-900 r/min).
12. Decant the plate immediately and remove residual liquid.
13. Pipette 1 ml of distilled water into all wells.
14. Incubate the plate for 5 minutes at room temperature on an orbital shaker (600-900 r/min).
15. Decant the plate immediately and remove residual liquid.
16. Pipette 100 μl of Hydrochloric Acid into all wells to elute the dopamine.
17. Cover the plate with adhesive foil and incubate for 10 minutes at room temperature on an orbital shaker (600-900 r/min). Caution: Do not decant the supernatant thereafter!

### Dopamine RIA

1. Pipette 90 μl of hydrochoric acid into the tubes for the NSB.
2. Pipette 90 μl of the extracted standards, controls and plasma samples into the respective tubes.
3. Pipette 25 μl of Enzyme Solution into all tubes (except totals).
4. Mix thoroughly and incubate for 30 minutes at 37° C.
5. Pipette 50 μl of the ¹²⁵I-Dopamine into all tubes.
6. Pipette 50 μl of Dopamine Antiserum into all tubes (except totals and NSB).
7. Mix thoroughly and centrifuge for 1 minute at 500×g
8. Incubate for 15-20 hours (overnight) at 2-8° C.
9. Mix the chilled (2-8° C.) Precipitating Reagent thoroughly, pipette each 1 ml into all tubes (except totals) and mix on a vortex.
10. Incubate for 15 minutes at 2-8° C.
11. Centrifuge for 15 minutes at 3,000×g, if possible in a refrigerated centrifuge.
12. Decant or aspirate the supernatant carefully (except totals). Beat out the tubes and leave it upside for 2 minutes.
13. Count all tubes for 1 minute in a gamma-counter.

### Calculation of Results

Concentration of the standards: Dopamine: A=0/B=9/C=30/D=90/E=300/F=900 pg/μl

Subtract the mean cpm of the non-specific binding NSB from the mean cpm of Zero Reference (=Standard A), Standards B-F, Control 1 & 2 and patient samples. Construct a standard curve by plotting the percentage of cpm of each standard in relation to the cpm of the Zero Reference ((B−NSB)/(B0−NSB) in %) versus its corresponding concentration. The concentration of the controls and patient samples can then be read off the standard curve by using their percentage of (B−NSB)/(B0−NSB).

### Quantitative Determination

The calibration curve from which the concentration of dopamine in the samples can be taken, is obtained by plotting % B/B0 values measured for the 6 standards (linear, y-axis) against the corresponding concentrations (logarithmic, x axis). The results for unknowns can be calculated using one of the following curve-fitting techniques: spline fits, Akima or four-parameter logistic.

### Results

### Reproducibility

The reproducibility of the RIA tests was investigated by determining the intra- and inter-assay coefficients of variation (CV) by repeated measurements of 3 plasma samples with different dopamine concentrations (see Table B1).

TABLE B1

| Intra-Assay Variation, n = 80 | | | Inter-Assay Variation, n = 15 | | |
|---|---|---|---|---|---|
| | Mean ± SD | | | Mean ± SD | |
| Sample | (pg/ml) | CV (%) | Sample | (pg/ml) | CV (%) |
| 1 | 31 ± 3.6 | 11.6 | 1 | 42 ± 7.5 | 17.8 |
| 2 | 105 ± 11 | 10.5 | 2 | 75 ± 7.6 | 10.1 |
| 3 | 314 ± 39 | 12.4 | 3 | 400 ± 52 | 13 |

### Recovery

Increasing amounts of dopamine were added to a plasma sample. Each spiked sample was assayed. The analytical recovery of dopamine was estimated at eight different concentrations by using the theoretically expected and the actually measured values (see Table B2).

TABLE B2

| | | | Determined | |
|---|---|---|---|---|
| | | Spiked level | concentration | |
| Sample | pg/ml | [pg/ml] | [pg/ml] | Recovery % |
| 0 | — | 15 | — | — |
| W1 | 400 | 415 | 399 | 96 |
| W2 | 200 | 215 | 230 | 107 |
| W3 | 100 | 115 | 106 | 92 |
| W4 | 50 | 65 | 58.7 | 90 |
| W5 | 25 | 40 | 37.4 | 94 |
| W6 | 12.5 | 27.5 | 20.7 | 75 |
| mean [%] | — | — | — | 92.3 |

### Linearity

The linearity of the RIA was investigated using seven different dilutions of a serum sample with Serum Equalizing Reagent (see Table B3).

TABLE B3

| Sample | [pg/ml] diluted | [pg/ml] found | [%] Linearity |
|---|---|---|---|
| L1 | 415 | 402 | 97 |
| L2 | 215 | 201 | 93 |
| L3 | 115 | 96 | 83 |
| L4 | 65 | 64.5 | 99 |
| L5 | 40 | 42.1 | 105 |
| L6 | 27.5 | 31.6 | 115 |
| L7 | 15 | 15 | 100 |
| mean [%]: | | | 98.9 |

### Example C

### Radioimmunoassay for the Quantitative Determination of Serotonin

A quantitative determination of serotonin levels in a sample may be performed by radioimmunoassay using commercially-available kits, such as the ‘Serotonin RIA’ kit of Labor Diagnostika Nord GmbH & Co KG, Nordhorn, Germany (Catalogue No. BA-0900).

### Reagents and Equipment

### Reagents for the Sample Acylation

Standard A; Serotonin (0 ng/ml)
Standard B; Serotonin (20 ng/ml)
Standard C; Serotonin (60 ng/ml)
Standard D; Serotonin (200 ng/ml)
Standard E; Serotonin (600 ng/ml)
Standard F; Serotonin (2000 ng/ml)

### Control 1

### Control 2

### Acylation Buffer

### Acylation Reagent

### Reagents for the Radio Immunoassay

Serotonin Antiserum, from rabbit
¹²⁵I-Serotonin, activity <200 kBq
Precipitating Reagent, goat anti-rabbit serum in PEG phosphate buffer (mix thoroughly before use).

### Additional Materials and Equipment Required

Automatic pipettes for 5, 25, 50, 100, 250, 500 and 2000 X
Plastic tubes (polypropylene, polystyrene) and suitable rack
Centrifuge (preferable refrigerated) capable of at least 3,000×g
Suitable device for aspirating or decanting the tubes.
Vortex mixer
Gamma counter
Distilled water

### Sample Collection and Storage

### Serum

The usual precautions for venipuncture should be observed. Blood has to be centrifuged within one hour after collection. Hemolytic and lipemic samples should not be used. Serum can be stored up to 24 hours at 2-8° C. For a longer storage (up to 6 months) the samples must be frozen at −20° C. Repeated freezing and thawing should be avoided.

### Urine

It is possible to use spontaneous as well as 24-hours urine. The total volume of urine excreted during a 24-hours period should be collected and mixed in a single bottle containing 10-15 ml of 6 M hydrochloric acid as preservative. Avoid exposure to direct sun light. Determine the total volume and take an aliquot for the measurement. For patients with suspected kidney disorders the creatinine concentration should be tested, too. Urine samples can be stored at −20° C. for at least 6 months.

### Plasma

More than 98% of the circulating serotonin is located in the platelets and is released during blood clotting. Blood must be collected by venipuncture into plastic tubes containing EDTA or Citrate.

### Platelet-Rich Plasma (PRP)

To obtain platelet-rich plasma (PRP) the samples are centrifuged for 10 minutes at room temperature at 200×g. Transfer the supernatant to another tube and count the platelets.

Platelets

The platelet pellet is obtained by adding 800 μl of physiological saline to 200 μl of PRP (containing between 350,000-500,000 platelets/μl) and centrifugation (4,500×g, 10 minutes at 4° C.). The supernatant is then discarded. 200 μl of dist. water is added to the pellet and mixed thoroughly on a vortex mixer. This suspension can then be stored frozen for several weeks at <−20° C.

After thawing of the frozen samples, centrifuge at 10,000×g for 2 minutes at room temperature. 25 μl of the supernatants are used for the acylation reaction.

### Platelet-Free Plasma (PFP)

To measure serotonin in platelet-free plasma (PFP), an aliquot of the PRP is centrifuged at 4,500×g for 10 min. at 4° C. 100 μl of the supernatant is used for the acylation reaction. Platelet-free plasma can be stored at −20° C. for up to two weeks.

### Tissue Homogenates and Cell Culture Supernatants

Centrifuged tissue homogenates and cell culture supernatants may be used without special precautions. Please notice that some cell culture media may contain serotonin.

### Cerebrospinal Fluid (CSF)

### Storage: at −20° C.

### Saliva

### Storage: at −20° C.

### Assay Procedure

### Sample Preparation and Acylation

Allow reagents—with the exception of Precipitating Reagent—to reach room temperature. Number the assay tubes (polystyrene or polypropylene) accordingly, Duplicates are recommended. The use of glass tubes is not recommended for the assay.

### Serum, Urine, Platelets:

1. Pipette 25 μl of Standard A-F, 25 μl of Control 1 & 2 and 25 μl of serum, urine, platelets, tissue homogenates and cell culture supernatants into the respective tubes.
2. Pipette 250 μl of Acylation Buffer into all tubes.
3. Pipette 25 μl of Acylation Reagent into all tubes.
4. Mix thoroughly and incubate for 30 minutes at room temperature (approx. 20° C.).
5. Pipette 2 ml of distilled water into all tubes and mix thoroughly.

Take 25 μl of the prepared standards, controls and samples for the Serotonin RIA

### Cerebrospinal Fluid, Platelet-Free Plasma and Saliva:

1. Pipette 25 μl of Standard A-F, 25 μl of Control 1 & 2 and 100 μl of cerebrospinal fluid (CSF), platelet-free plasma (PFP) and saliva into the respective tubes.
2. Pipette 250 μl of Acylation Buffer into the tubes for standards and controls and 50 μl into the tubes for CSF, PFP and saliva.
3. Pipette 25 μl of Acylation Reagent into the tubes for standards and controls and 5 μl into the tubes for CSF, PFP and saliva.
4. Mix thoroughly and incubate for 30 minutes at room temperature (approx. 20° C.).
5. Pipette 2 ml of distilled water into the tubes for standards and controls and 300 μM into the tubes for CSF, PFP and saliva and mix thoroughly.

Take 25 μl of the prepared standards, controls and samples for the Serotonin RIA

### Serotonin RIA

1. Pipette 25 μl of prepared Standard A into the tubes for the NSB.
2. Pipette 25 μl of prepared Standards A-F, Controls 1 & 2 and patient samples into the respective tubes.
3. Pipette 50 μl of ¹²⁵I-Serotonin into all tubes.
4. Pipette 50 μl of Serotonin Antiserum into all tubes (except totals and NSB).
5. Mix thoroughly and centrifuge for 1 minute at 500×g.
6. Incubate for 90 minutes at 2-8° C.
7. Pipette 500 μl of the chilled (2-8° C.) Precipitating Reagent into all tubes (except totals) and mix on a vortex.
8. Incubate for 15 minutes at 2-8° C.
9. Centrifuge for 15 minutes at 3,000×g, if possible in a refrigerated centrifuge.
10. Decant or aspirate the supernatant carefully (except totals). Beat out the tubes and leave it upside for 2 minutes.
11. Count all tubes for 1 minute in a gamma counter.

### Calculation of Results

Subtract the mean cpm of the non-specific binding NSB from the mean cpm of Zero Reference (=Standard A), Standards B-F, Control 1 & 2 and patient samples. Construct a standard curve by plotting the percentage of cpm of each standard in relation to the cpm of the Zero Reference ((B−NSB)/(B0−NSB) in %) versus its corresponding concentration. The concentrations of the controls and patient samples can then be read off the standard curve by using their percentage of (B−NSB)/(B0−NSB).

The read concentrations of the platelet-free Plasma, saliva and the Cerebrospinal fluid have to be divided by 20.

### Calculation of Serotonin in Platelets

The content of serotonin in platelets is referred to 10⁹ platelets. Following is given an example:

Serotonin concentration: 100 ng/ml

Number of the platelets in the PRP: 300.000/μl=0.3×10⁹ platelets/ml with serotonin content of 100 ng.

The resulting serotonin content in the platelets is 333 ng/10⁹ platelets (100 ng serotonin×1.0×10⁹/0.3×10⁹)

### Quantitative Determination

The calibration curve from which the concentration of serotonin in the samples can be taken is obtained by plotting the % B/B0 values measured for the 6 Standards (linear, y-axis) against the corresponding concentrations (logarithmic, x-axis). The results for unknowns can be calculated using one of the following curve-fitting techniques: spline fits, Akima or four-parameter logistic.

### Example D

### Radioimmunoassay for the Quantitative Determination of Melatonin

A quantitative determination of melatonin levels in a sample may be performed by radioimmunoassay using commercially-available kits, such as the ‘Melatonin Reseach RIA’ kit of Labor Diagnostika Nord GmbH & Co KG, Nordhorn, Germany (Catalogue No. BA-3900).

### Reagents and Equipment

### Reagents for the Radio Immunoassay

Standard A, Melatonin (0 pg/ml)
Standard B, Melatonin (30 pg/ml)
Standard C, Melatonin (100 pg/ml)
Standard D, Melatonin (300 pg/ml)
Standard E, Melatonin (1000 pg/ml)
Standard F, Melatonin (3000 pg/ml)
Standard G, Melatonin (10000 pg/ml)

### Control 1

### Control 2

### Assay Buffer

Enzyme, lyophilized, redissolve in Enzyme Buffer

### Enzyme Buffer

Melatonin Antiserum, from rabbit
¹²⁵I-Melatonin, activity <200 kBq
Precipitating Reagent, 55 ml, goat anti-rabbit serum in PEG phosphate buffer.

### Additional Materials and Equipment Required

Automatic pipettes for 25, 50, 100 and 500 μl
Plastic tubes (polypropylene, polystyrene) and suitable rack
Centrifuge (preferable refrigerated) capable of at least 3,000×g
Suitable device for aspirating or decanting the tubes.
Vortex mixer
Gamma counter
Distilled water

### Sample Collection and Storage

The test can be performed with EDTA plasma as well as with heparin plasma and serum.

The plasma samples can be stored up to 24 hours at 2-8° C. For a longer period (up to 6 months) the samples should be stored at −20° C. Repeated freezing and thawing should be avoided.

Sample volumes of 400 μl were used.

### Assay Procedure

### Preparation of Reagents

Plasma standards: Reconstitute Standard A (2.5 ml) with 2 ml distilled water, Standards B-F each with 1 ml. Reconstituted standards which are not used immediately have to be frozen at −20° C. (in aliquots) and may be thawed only once.

Controls 1 & 2: Reconstitute the controls each with 1 ml distilled water. Reconstituted controls which are not used immediately have to be frozen at 20° C. (in aliquots) and may be thawed only once.

Enzyme: Reconstitute the content of the vial with 3 ml Enzyme Buffer prior to use. Mix carefully (30 minutes on a rotating mixer), The reconstituted enzyme cannot be stored and has to be used only once.

### Melatonin RIA

Allow reagents and samples with the exception of Precipitating Reagent—to reach room temperature. Number the assay tubes (polystyrene) accordingly. Duplicates are recommended.
1. Pipette 20 μl of Standard A into the tubes for the NSB.
2. Pipette 20 μl of Standards A-G and controls into the respective tubes.
3. Pipette 400 μl of Equalising Reagent into tubes with NSB, Standards A-G and controls.
4. Pipette 400 μl of sample into the respective tubes.
5. Pipette 50 μl of Enzyme solution in all tubes (except totals), vortex and centrifuge 1 minute at 500×g.
6. Incubate for 1 hour at room temperature (approx. 20° C.).
7. Pipette 100 μl of Assay Buffer into all tubes (except totals) and mix shortly.
8. Pipette 50 μl of Melatonin Antiserum into all tubes (except totals and NSB).
9. Mix thoroughly and centrifuge for 1 minute at 500×g.
10. Incubate for 1 hour at room temperature without shaking
11. Pipette 50 μl of ¹²⁵I-Melatonin into all tubes.
12. Mix thoroughly and centrifuge for 1 minute at 500×g.
13. Incubate for 20 to 24 hours at room temperature (approx. 20° C.) without shaking.
14. Mix the chilled (2-8° C.) Precipitating Reagent thoroughly and pipette 1000 μl into all tubes (except totals) and mix on a vortex.
15. Incubate for 20 minutes at 2-8° C.
16. Centrifuge for 20 minutes at 3,000×g, if possible in a refrigerated centrifuge.
17. Decant or aspirate the supernatant carefully (except totals). Beat out the tubes and leave it upside for 2 minutes.
18. Count all tubes for 1 minute in a gamma counter.

### Calculation of Results

Subtract the mean cpm of the non-specific binding NSB from the mean cpm of Zero Reference (=Standard A), Standards B-G, Control 1 & 2 and patient samples. Construct a standard curve by plotting the percentage of cpm of each standard in relation to the cpm of the Zero Reference ((B−NSB)/(B0−NSB) in %) versus its corresponding concentration. The concentrations of the controls and patient samples can then be read off the standard curve by using their percentage of (B−NSB)/(B0−NSB).

### Quantitative Determination

The calibration curve from which the concentration of melatonin in the samples can be taken is obtained by plotting the % B/B0 values measured for the 6 standards (linear, y-axis) against the corresponding concentrations (logarithmic, x-axis). The results for unknowns can be calculated using one of the following curve-fitting techniques: spline fits or Akima.

### Example E

### Study of Levels of Dopamine, Serotonin and Melatonin in Cushingoid/Laminitic Horses and Matched Healthy Horses

### Introduction

Equine Cushing's syndrome is usually defined as systemic hypercorticism. Several variants of the disease have been described (1) but the most widely recognised is pituitary pars intermedia dysfunction (PPID) (2) in which hypertrophy and hyperplasia of the pituitary pars intermedia are often observed. The resulting clinical signs may include hirsutism, polydipsia, polyuria, hyperhydrosis, protein catabolism (decreased muscle mass), episodes of laminitis, glucose intolerance and insulin refractoriness, suppression of the immune system and general lethargy. Cushing's syndrome is a progressive disease mainly affecting aged horses and ponies (>16 years) but has been recognised in individuals as young as 7 years. There does not appear to be a gender predisposition, but pony breeds, Morgan horses and domesticated Spanish Mustangs are over-represented in some epidemiological studies. The etiology and pathogenesis of the condition are incompletely understood but thought to derive from degeneration of the periventricular hypophyseal dopaminergic neurons and concomitant loss of dopaminergic inhibition of POMC-derived peptides (3). Prognosis tends to be associated with the prevalence of laminitis in the individual as the effects of this clinical sign are usually most severe. The onset of laminitis associated with PPID is most often observed in the autumn (August-October inclusive).

Equine laminitis has been re-defined in recent years as a systemic disease, which manifests as a condition of the hoof (4, 5). It is a debilitating and extremely painful situation in which there is disrupted haemoperfusion of the hoof and the attachment between the third phalanx (P3) and supporting internal tissue is thought to become enzymatically degraded (6). This results in inflammation, ischaemic-reperfusion injury and varying degrees of subsequent soft tissue necrosis plus displacement and/or remodelling of the P3. The disease may occur in any number of hooves as an acute, transient episode or as a chronic condition with variation in degree of severity, including sub-clinical states. Events acknowledged to trigger the onset include hoof concussion from exercise, ingestion of materials providing a fermentable substrate for hindgut bacteria (7, 8), toxaemia or other conditions of poisoning, iatrogenic therapies (particularly cortisone administration), stress (such as road transport) or as secondary to other conditions such as gastrointestinal disorders. Any horse, of any age, may develop the disease but pony breeds and “good doers” are known to be particularly susceptible; again, the etiology and pathogenesis are incompletely understood. As mentioned above, there appears to be a strong link between the incidence of laminitis and PPID. Although it is probable that any systemic disease may increase susceptibility to laminitis, laminitis is acknowledged to be a clinical sign of PPID and the main reason for euthanasia being carried out in advanced cases. A recent epidemiological study (9) has provided corroboration of the general practice view that laminitis is one of the most common equine diseases: in surveys carried out in both the USA and the UK it was found that around 3% of the equine population might be expected to experience acute laminitis per annum. Aside from the obvious animal welfare concerns, laminitis is also of economic importance (9, 10) having significant financial impact upon owners of horses. Its prognosis is often difficult to predict, as an individual case may result in temporary compromise of athletic ability, permanent changes to hoof physiology, the animal unable to work or, in severe cases, euthanasia, due to the degree of lameness and associated level of pain. Susceptibility to developing the disease is greatest in the Spring (February-April inclusive) and/or Autumn (August-October inclusive), tending to coincide with seasonal flushes of grass in temperate western countries.

Currently, the causes, progression and treatment of independently occurring laminitis, PPID associated laminitis and PPID itself are hotly debated by researchers and there are no reliable preventatives or remedies for the conditions (5). Treatments are generally given to ameliorate the clinical signs and may include dopamine agonists, serotonin antagonists and/or non-steroidal anti-inflammatory drugs and blood vessel dilators. In the United Kingdom, there are no licensed veterinary pharmaceuticals which may be applied to cases of equine laminitis or PPID. Thus, not only are the majority of administered drugs only approved for human use, their mode of action in the horse is poorly understood.

However, the relevance of neurohormones to these conditions is now starting to be recognised. Study of vaso-active amines with molecular forms similar to the precursors or actual forms of serotonin and/or dopamine has been carried out, in the context of laminitis (11, 12). Examination has also been made of Cushing's syndrome in a variety of forms, including the degradation of dopaminergic neurons (3, 13). Nycthemeral change in serum tryptophan and serotonin has been investigated (14) and there is some disagreement regarding the relevance of melatonin in equine reproductive cycles (15, 16), but such studies do provide a framework to investigate the effects of equine neurohormone daily rhythms and seasonality more closely.

Despite this work, there is still no accepted, unifying theory linking the putative triggers of laminitis or causal explanation as to why laminitis may be a clinical sign of PPID. The pathological mechanisms behind increased laminitis susceptibility in the spring and autumn for horses in general and, in cases of PPID, increased likelihood of autumnal laminitis onset are unclear. We believe that these mechanisms may be better understood in the context of gerontological changes over the lifetime of an individual:

In human medicine, changes or imbalances within the endocrine system are now being accepted as contributory to age-related conditions and diseases. One such condition is Metabolic Syndrome (Reaven's syndrome) (17, 18), also known as Abdominal Obesity Metabolic Syndrome, in which a number of signs and symptoms are observed due to insulin resistance and resulting hyperinsulinemia. There is a suggestion that this may be prodromal to Cushing's syndrome (19) and it has been proposed that a similar condition is also found in horses (1, 20). What does not appear to have been considered is the possible link to these diseases from events within the chain of cascade of metabolic control, i.e. to neuroendocrine considerations.

The Neuroendocrine (or Ontogenetic) Theory of Aging (21) provides a framework to address this. The origin of disease is described as having three models: ecological (external factors), genetic and accumulational, with all such diseases having common pathogenetic factors, e.g. hyperinsulinemia and resistance to the inhibitory effects of dexamethasone upon the secretion of corticosteroids. Through the growth, development, and subsequent aging of an individual, a loss of central and peripheral neurotransmitter and hormone sensitivity occurs gradually, over time, and causes a progressive shift in homeostasis throughout an individual's life. The result is hormonal and metabolic shifts that are causal in aging and the “diseases of aging” (22, 23, 24). It has been proposed that the root of these effects is slow degradation and/or atrophy of the pineal gland (25) and progressive cumulative decrease in the indole amines it produces: serotonin and melatonin (26, 27). However, there is another very important relationship which changes during aging: if a graph of serotonin and dopamine decline is plotted against years (21), the rate of decrease of the catecholamine, dopamine, is found to be much greater than that of serotonin. The pineal gland is thought to be implicated in this situation through the suggestion that melatonin sensitises and entrains dopamine synthesis/release rhythm (28, 29).

In humans, as with horses, it is often found that these so called “diseases of aging” (for example Diabetes Mellitus—Type Two DM2) in humans, or laminitis in horses) may occur at any age (30), even though there may be a greater prevalence in older individuals. When all parts of an individual are considered as having not only a chronological age, but also a biological age, this discrepancy is more easily understood (21). In this document we are describing “biological age” as the state of a specific part of the body compared to its expected condition in terms of chronological years. For example, a fifteen year old human may show clinical signs of DM2, or a five year old horse may contract laminitis, due to obesity. We propose that it is not the state of excess adipose tissue per se that causes the clinical signs, but physiological changes and a systemic change in neuroendocrinology, which may mimic, however transiently, the internal environment found in a chronologically older individual. Prior to manifestations of symptoms or clinical signs, respectively, the human might be considered to have a pancreas in the same condition as a healthy, but older human, and the horse might be considered to have hoof physiology nearer to that of an older horse. Thus, the pancreas and the hoof may be observed to be of a greater biological age, than the chronological age of the individual.

In summary, the progressive degeneration of the pineal gland is thought to cause change in the relative ratios of serotonin, melatonin and dopamine over time. This results in the manifestations of biological aging. Also, a transient aged state may develop at any point in time, dependent upon the combination of inherent genetics and external factors, resulting in the same pathologies. For example, there are shared endocrinopathies in horses with PPID (20, 21) and cases of acute carbohydrate-overload laminitis (22, 23, 24); a combination of the following may be in attendance in both scenarios: hypercorticism, hyperinsulinemia, hyperglycaemia, glucose intolerance and hypertension. The broad aims of this study were to investigate whether the Neuroendocrine Theory of Aging was a valid context to examine the pathology of equine Cushing's syndrome and whether it might provide a new approach to investigate the causal mechanisms of laminitis susceptibility Specifically, we have examined the hypothesis of a link between the presence or absence of the clinical signs of PPID and differential peripheral plasma concentrations of serotonin, melatonin and dopamine within aged horses and ponies.

### Materials & Methods

### Animals

A population of test animals which represented a random selection of breeds or types of horse and pony from varied geographical locations throughout the United Kingdom. This population was static, in that its members remained at the site throughout their lives and new individuals were introduced on an infrequent basis. The horses and ponies selected for the study were aged between 21 and 36 years, had been retired from work and were kept at grass. However, they were brought into stables from 16:00 to 07:30 during the December and March sampling sessions. This regime was started well in advance of the session (a minimum of 4 weeks) for most individuals as part of their regular winter management, with any exceptions being documented. The study was conducted without making any changes to the animals' normal feeding and management regime which was applied consistently over the course of the study. All experimental protocols were carried out in strict accordance with United Kingdom Home Office regulations.

### Experimental Strategy and Design

Two groups were constructed from the population: a “disease” group and a “control” group. The disease (or PPID) group was selected first and consisted of horses and ponies with a veterinary history which strongly implied the presence of PPID, or where external examination confirmed the same. The control group was then selected from the same population with each member matching an individual in the disease group as closely as possible; that is, on the following variables: breed/type, gender, weight and height. A total of thirty animals, fifteen in each group, was initially proposed. To diagnostically confirm that each individual was in the correct group, static determinations were made of the following substances from a blood plasma or serum sample obtained via jugular venepuncture from each animal: ACTH, cortisol, insulin, glucose, triglycerides, total cholesterol, magnesium and alkaline phosphatase. Standard normal ranges were applied in clinical consideration of each individual. From the starting pool of thirty individuals, twelve matched pairs of horses and/or ponies which best represented the two states (disease and control) to be compared were put forward for the study. Individuals with a good chance of survival to the end of the study and negligible anxiety during jugular venepuncture were considered to be the best candidates.

Blood samples were collected at 27-hour intervals over 4×10 day periods, each scheduled to span consecutive solstices and equinoxes throughout a year. Thus, sampling period 1 ran from the 20 Jun. 2005 to 29^{th} June 2005 inclusive, sampling period 2 ran from 19^{th} September 2005 to 28 Sep. 2005 inclusive, sampling period 3 ran from 12^{th} December 2005 to 21 Dec. 2005 inclusive and sampling period 4 ran from 13^{th} March 2006 to 22^{nd} Mar. 2006 inclusive. Day 1 commenced at 13:00 and was a control for Day 9, also at 13:00. All sampling during hours of darkness was done using dim red light to minimise effects on the pineal gland. Different operators were used to draw samples from different horses across both groups to reduce bias. If the horses and ponies were at grass, they were caught and brought into catch-up areas in the hour preceding the sampling start time; each individual was secured and given a minimum of 20 minutes in a calm. state before sampling commenced. The samples were then collected via needle and syringe draw from the jugular vein, gently transferred to BD Vacutainer® spray-coated K₂EDTA plastic tubes (without using the vacuum, i.e. not through the cap) and kept within insulated boxes at between 2° C. and 8° C. They were promptly centrifuged at 1300G for 12 minutes within 2 hours of collection, immediately processed and stored short-term between −25° C. and −30° C. Transportation to the laboratory was within a week of the close of each sampling session and all samples were professionally packed, on dry ice, in a purpose made container for data-logged carriage at between −30° C. and −50° C. Long term storage at the laboratory was at −75° C.

To also be able to examine individual circadian profiles, the experimental design included a consistent time slot for each horse or pony on each sampling day. For example, Horse 1 had a sample drawn at 13:00 on Day 1. This individual would then have a sample drawn at 16:00 on Day 2. Similarly, Horse 2 had a sample drawn at 13:06 on Day 1. This individual would then have a sample drawn at 16:06 on Day 2. Thus, the time slots were 6 minutes apart; they were maintained for each individual over the course of the study. Because the purpose of this study was to observe the natural rhythms of the horses, but also to contend with the UK yearly shifts between GMT and BST (thus, adaptation of the horses to management and feeding changes associated with this) all sampling times are as would be expected in the UK, i.e. GMT for December and March, BST for June and September.

### EDTA Plasma Radio-Immuno Assays (RIA)

Commercial ¹²⁵I RIA kits were obtained for quantitative determinations of serotonin, melatonin and dopamine (Labor Diagnostika Nord GmbH & Co, KG, 48531 Nordhorn, Germany). These kits were validated for use with equine plasma via standard parallel and serial dilution techniques and also by running test samples at the same concentrations within the same assay kit and across different assay kits of the same product code. The manufacturer reported that analytical sensitivity was 10 ng/ml, 0.4 pg/ml (400 μl version) and 6 pg per sample volume unit extracted (ml) for the serotonin, melatonin and dopamine kits, respectively. The assay procedures followed the basic principle of radio-immuno assays, involving competition between a radioactive and a non-radioactive antigen for a fixed number of antibody binding sites. The amount of ¹²⁵I-labeled antigen bound to the antibody was inversely proportional to the analyte concentration of the sample. When the system was in equilibrium, the antibody bound radioactivity was precipitated with a second antibody in the presence of polyethylene glycol. The radioactivity of the precipitate was then measured in a gamma counter. Quantification of unknown samples was achieved by comparing their activity with a reference curve prepared with known standards.

To prepare the samples for serotonin determination, an acylation reagent was used to quantitatively derivatise the serotonin into N-acylserotonin. To prepare the samples for melatonin determination, an “Equalising Reagent” was used to counter the unpredictable influence of complex proteins and peptides on assay performance. This reagent was produced by use of the respective biological liquid; in this case, equine plasma. Endogenous melatonin was removed by adsorption to activated charcoal and the melatonin-free biological liquid was then used to equalise the assay matrix of standards and untreated samples. Finally, all samples, including the standards, were digested by use of a protease to reduce non-specific binding, for example to albumin or albumin-like molecules. To prepare the samples for dopamine determination the dopamine was extracted using a cis-diol specific affinity gel, acylated to N-acyldopamine and then converted enzymatically during the detection procedure into N-acyl-3-methoxytyramine.

Further testing of the dopamine kit was carried out to confirm its reliability in determining the low volumes of dopamine in equine plasma. The manufacturer provided the following additional information from their own quality control testing using equine EDTA plasma: reproducibility of the RIA tests was investigated by determining the intra-assay and inter-assay coefficients of variation (CV) by repeated measurements of 3 plasma samples with different dopamine concentrations. This resulted in an intra-assay CV range from 10.5% to 12.4% and an inter-assay CV range from 10.1% to 17.8%. Recovery was investigated by adding increasing amounts of dopamine to a plasma sample. Each spiked sample was assayed. The analytical recovery of dopamine was estimated at eight different concentrations by using the theoretically expected and the actually measured values. This resulted in a mean recovery of 92.3%. Linearity was investigated using seven different dilutions of a serum sample with Serum Equalizing Reagent, which resulted in a mean linearity value of 98.9%.

Further testing of the melatonin kit was also completed at the volume used for determination of the samples from the study (400 μl). The manufacturer provided the following additional information from their own quality control testing using equine EDTA plasma: reproducibility of the RIA tests was investigated by determining the intra-assay and inter-assay coefficients of variation (CV) by repeated measurements of 3 plasma samples with different melatonin concentrations. This resulted in an intra-assay CV range from 4.2% to 19.7% and an inter-assay CV range from 9.4% to 22.0%. The analytical recovery of melatonin was estimated at seven different concentrations by using the theoretically expected and the actually measured values. This resulted in a mean recovery of 100.2%. Linearity was investigated using seven different dilutions of a plasma sample, which resulted in a mean linearity value of 84.9%.

Prior to assay, the samples were left on the bench to thaw at room temperature (approximately 20° C.) until the ice had melted, immediately centrifuged at 6° C. and 3000 G for 5 minutes to remove any particulates, then immediately used in the assay. The assay kit protocols, timescales and quality control measures were strictly adhered to. As far as possible, all samples for the same horse or pony were assayed at the same time in the same kit for each sampling period. Each sample was numbered to remove opportunity for operator bias during the assay procedures.

### Statistical Analyses

The Resource Equation method was initially used to establish the size of the groups of horses and ponies taking into consideration the constraints of logistics, size of establishments housing suitable populations of horses and cost-benefit analysis.

Analysis of variance via split-plot ANOVA was then employed to search for statistically significant differences in neurohormone concentrations between the control and PPID groups. In the first instance, it was assumed that a single analysis of variance could be carried out within each season for each of the following measures: serotonin, melatonin and dopamine concentrations and serotonin:melatonin, dopamine:melatonin and serotonin:dopamine concentration ratios (the ratio directions were selected to minimise the effect of zero or missing data in the denominators). Due to the natural variation in neurohormone concentrations in individual horses and the diurnal variation in magnitude of the neurohormones (particularly melatonin), further analyses were also made for these measures, to examine the effects of, and interaction between, condition (or “treatment”—horse group), season, time and dark and light periods. Data were available for approximately half of the samples collected during the fieldwork sessions, i.e. 6 PPID horses with their control matches. All analyses were carried out by QI Statistics Ltd, Reading, UK using the SAS software package.

Firstly, the control days were compared using the difference in adjusted mean scores between Time 1 (13.00 Day 1) and Time 9 (13.00 Day 9) for each “treatment” group via t-test.

A 3-way split plot analysis of variance for repeated measurements over time was then carried out, fitting the following effects: Month¹, Condition¹, Horse¹, Month*Condition¹, Horse*Month*Condition (main plot error), Time, Time*Month, Condition*Month, Condition*Time*Month. The terms is marked¹ were tested against the main plot error, the remaining terms against the split plot error. This analysis was used to identify outliers and to check the distribution of the residual variability. It was assessed graphically as reasonably normally distributed but showed some extreme outliers; 3 such outliers were removed. The 3-way split plot ANOVA was then rerun omitting the outliers. This was followed by analysis of variance of the data separately for each month, fitting the following effects: Condition, Horses within Condition (MP Error), Time and Condition*Time; Time and Time by Condition were tested against the split plot error A comparison of “treatments” by time was then made using t-test pair-wise comparisons of the differences in adjusted means at each time point. These comparisons were carried out using adjusted means to take account of instances where entries were missing from the dataset; due to a horse not being available, for example.

Finally, an analysis was made of the light and dark periods separately. Similarly to the procedures above, a comparison of “treatments” by time was made using t-test pair-wise comparisons of the differences in adjusted means at each time point. The photophase and scotophase were defined using the sunrise and sunset times for the latitude and longitude of the site.

Because this study was a novel investigation of PPID, statistical results were reported up to an alpha level of 0.15; otherwise NS (“Not Significant”) was returned. Results with an alpha level between 0.15 and 0.10 were considered to be pointers to areas of interest for further study. Results with an alpha level between 0.10 and 0.05 were considered to be worthy of clinical interest. Results with an alpha level between 0.05 and 0.00 were considered to be of most clinical interest. All graphical data are presented as adjusted means ±SEM.

### Results

### Statistical Analysis

Detailed statistical analyses are provided in Examples F and G below.

Overall, there was no strong evidence of a significant difference between the average neurohormone concentrations on each of the two control days for any of the seasonal sampling periods. Thus, it was felt reasonable to assume that the result on each sequential day was reliable.

The split-plot ANOVA of time by treatment interaction (group) by month returned the following significant differences (p-values for F Test): 0.0778 for dopamine, <0.0001 for serotonin:melatonin and <0.0001 for dopamine:melatonin, When this was broken down via analysis of variance for each month separately, the following significant differences were found for the time by treatment interaction (group) effect (p-values for F Test): in June, 0.0773 for dopamine and 0.1204 for serotonin:dopamine; in September, 0.04137 for dopamine and 0.1288 for serotonin:melatonin; in December, <0.0001 for serotonin:melatonin and 0.0003 for dopamine:melatonin; in March, 0.0456 for dopamine. The t-test pair-wise comparisons of the differences in adjusted means at each time point, analysed by grouping the data by photophase and scotophase, for each neurohormone and the neurohormone ratios are displayed in Tables G4 to 07 in Example G, below). These are followed by circadian rhythm graphs depicting PPID and control group neurohormone concentrations, by sampling period (from t-test pair-wise comparisons of the differences in adjusted means at each time point, analysed by grouping the data by photophase and scotophase), shown in FIGS. 1 to 3. Graphs compiled similarly for the statistically significant neurohormone ratio concentrations, via analysis of variance for each month separately, are shown in FIGS. 4 to 6.

### Comparison of PPID and Control Groups: Neurohormone Rhythms

The dopamine profiles in PPID and control horses appear to be broadly biphasic, with similar duration of plasma concentration increase/decrease and amplitude of rhythm in March and December (see FIG. 1). However, a variable decrease in plasma dopamine concentrations is consistently shown in PPID horses across the year; this is most pronounced in June and September with a phase shift around the onset of scotophase, compared to controls, i.e. this dopamine peak is later in time in June and earlier in time in September for PPID individuals. The nocturnal rise in plasma melatonin concentrations appears to be broadly of the same duration and of similar amplitude in horses suffering from PPID and in controls (see FIG. 2). However, a small and somewhat variable increase in plasma concentrations is seen in PPID horses, across the year, with a minor acrophase shift between the groups in June and more pronounced acrophase shift between the groups in March. There is also evidence of a stepped acrophase in March and December for both groups. The serotonin rhythms for PPID and control horses are broadly biphasic, across the year, with similar duration of plasma concentration increase/decrease but lower rhythm amplitude in March for PPID horses compared with controls (see FIG. 3). However, in June, the controls appear to have a more pronounced rhythm with increased duration of plasma concentration rise (greater number of peaks) and increased acrophase amplitude, compared to PPID individuals; both rhythms are quite different. In September, both groups show a somewhat similar duration of plasma concentration increase/decrease and amplitude of rhythm, but in PPID horses there is increased amplitude at acrophase which is shifted to the left during photophase and shifted to the right during scotophase, compared to controls. A similar situation exists in December, with both groups following a broadly similar rhythm in terms of duration of plasma concentration increase/decrease and rhythm amplitude; however, there is a variable decrease in plasma concentrations for PPID individuals across the sampling period with acrophase shifted to the left, compared to controls.

### Comparison of PPID and Control Groups: Neurohornone Ratios

The plasma neurohormone concentration ratios are well represented in the analysis of statistically significant differences between the PPID and control groups. Comparison was made of circadian rhythm graphs depicting PPID and control group neurohormone ratio concentrations, by sampling period (from t-test pair-wise comparisons of the differences in adjusted means at each time point, analysed by grouping the data by photophase and scotophase) and the tables of significant differences produced via the same mechanisms to ascertain where the strongest signals of difference might lie. It was discovered that there are pronounced peaks in the graphs; the most obvious directly corresponding back to the significant differences made apparent by the analysis of variance (see FIGS. 4 to 6). For serotonin:melatonin and dopamine:melatonin, there are photophase signals in September (acrophase at 16:00) and December (acrophase at 10:00), with the December signal being most pronounced. For serotonin:dopamine, there are strong signals in photophase (19:00) and in scotophase (22:00) in June; additionally, there is a weaker signal for this ratio in scotophase (01:00) in September.

### Discussion

In this study, we have sought to provide evidence of the relevance of an imbalance of serotonin, melatonin and dopamine to the clinical signs of pituitary pars intermedia dysfunction in aged horses and ponies. We believe that these findings demonstrate that PPID in the horse develops as a result of a cumulative, progressive imbalance of serotonin, melatonin and dopamine. Also, that any form of laminitis, whether independently occurring or not, results from the same imbalance but in an acute, transient sense. This newly identified cause for laminitis may have ramifications in the broader context of equine gerontology (including increasing susceptibility to laminitis with time); for example, in consideration of insulin resistance and glucose intolerance in the horse including equine metabolic syndrome (1) and the recently proposed “pre-laminitic metabolism syndrome” (36).

Because much recent work has used experimental models of laminitis (thus, observable in vivo or in vitro changes may not precisely mirror the naturally occurring disease), the present study was designed to examine the horse in its natural state as closely as possible, with practical applications of the research in mind. In the first instance, although the fieldwork has produced separate platelet poor and platelet rich samples for serotonin determinations, it was thought important to investigate the total concentration of serotonin in an EDTA blood plasma sample as might be easily procured by a veterinary practitioner. Examination of serotonin systems in the horse, particularly the significance of platelets, is thought to be a useful direction in studying the pathogenesis of laminitis (11, 37, 38, 39); how and when the serotonin bound in platelets is released into the circulatory system may be intimately involved in the onset of the disease. The results of this study therefore concern determinations of total serotonin, melatonin and dopamine concentrations in equine plasma, from the perspective that free serotonin circulating in the bloodstream, at a given time and in contact with the various body systems, may change to any concentration up to the total concentration present. Additionally, it is known that plasma concentrations of dopamine and melatonin are low in many mammalian species (pg/ml); a key part of this study was the successful validation of reliable dopamine and melatonin RIA's with the required sensitivity for equine determinations. There are few examples of equine dopamine rhyhms in the literature, presumably because it is only recently that assay technology has sufficiently progressed for accurate measurement.

We have confirmed that the clinical manifestations of PPID appear to be associated with both circadian and circannual changes in circulatory neurohormonal concentrations. Little is known about the physiological regulation of peripheral neurohormone secretion into the circulation and the significance of this circulatory presence in the horse; theories concerning the pathogenesis of laminitis per se have tended to focus on these types of substances from a less intrinsic point of view, e.g. as end-result products from hind-gut bacterial populations or effects stemming from such events. For example, plasma dopamine concentrations might either parallel the changes in noradrenaline or adrenaline concentrations, on their way to elimination from the body, or they may reflect a specific dopaminergic effect. Thus, changes in plasma neurohormone concentrations may play a role only as a marker of peripheral neurohormone systems activation or act as a primary mediator of metabolic effects (42). We believe that this study provides evidence to support the latter role in cases of PPID. There is precedent for this view from observations such as the intravenous infusion of individual amines into normal horses, decreasing blood flow to the digits (43). It is also well known that stimulation of central autonomic pathways causes vasodilation in the canine paw pad (42); some studies of equine laminitis report an initial vasodilation event. Although the horse's hoof is a very different structure, it may prove valuable to investigate the level of dopaminergic peripheral innervation that is present.

From a chronological perspective obviously both groups of horses were aged (>16 years), but from the viewpoint of the Neuroendocrine Theory of Aging (21) the PPID group might be considered to be of greater “biological age” than the control group. Serotonin and dopamine plasma concentrations appeared generally decreased throughout the year in PPID individuals, compared to controls. There was also a pronounced dopamine rhythm phase shift and amplitude decrease for the PPID group in June and September, in addition to evidence of serotonin acrophase amplitudes being increased in September. These observations are consistent with the reduction in dopamine and concomitant increase in serotonin implied in the treatment of PPID horses with dopamine agonists and serotonin antagonists. Of particular interest is the apparent correlation between these results and the annual changes in severity of the clinical signs associated with PPID: anecdotal evidence from owners and veterinary practitioners has indicated that the condition of Cushingoid horses may begin to worsen in the summer, leading to laminitis in the autumn, with recovery in the winter and a brief decrease in clinical signs (for some) in the spring. Although our results do not show a marked increase or decrease in melatonin plasma concentrations in the PPID group, as compared to controls, the acrophase shift and increased amplitude in March holds potential for investigating why many Cushingoid horses do not shed their winter coat in the normal way (as would be expected at this time of year). This may also apply to the subsequent increases in follicle density and length plus coarsening of the hair structure that have been observed. It is worth noting that the significant differences between the groups in our results appear to pre-date the traditionally acknowledged seasons (spring, autumn) of increased laminitis susceptibility by approximately three months (as well as being present during such seasons); this is perhaps not when most practitioners would expect them. We believe that this demonstrates a time lag between the body state becoming relatively more imbalanced (in a circannual neuroendocrine sense) and progression to a critical point, beyond which laminitis develops. The situation may, of course, be exacerbated by other trigger factors for laminitis becoming part of the external conditions to which the horse is exposed, e.g. access to a flush of spring grass. Additionally, it might be expected that dopamine and serotonin would be the dominant factors implicated in investigating PPID, as defined by past treatment regimes. However, when the results of the present study were examined as a whole, taking into account assay sensitivity, the neurohormone rhythms that were discovered and neurohormone rhythms that exist in the literature for healthy horses (14, 40, 41), the signals felt to be most reliable were dopamine plasma concentrations and serotonin:melatonin and dopamine:melatonin plasma concentration ratios, with serotonin:dopamine only represented for two times in June (as shown in FIG. 6). This may be because we have examined total plasma serotonin; the comparison with platelet poor samples will certainly be interesting and a different relationship from this latter type of sample may indicate the importance of platelet uptake and release of serotonin. It is also possible that there is a somewhat increased conversion rate of serotonin to melatonin in PPID individuals, compared to controls, during most of the year (excluding September), but this is a topic for further study. We have anecdotal evidence to support dopamine and melatonin being important in the pathogenesis of PPID: it appears that when a low dosage of melatonin (1.5 mg) is given consistently, in the evening each day, all year round, that this facilitates shedding of the winter coat at the expected time and a much more normal appearance. Additionally, changing the time of day at which pergolide is administered, changing the dosage (by as little as 0.05 mg per day) and under or over dosing relative to the physiological requirements of the individual appears to increase the probability that laminitis will occur; 0.75 mg may be a maximum dosage for ponies. Previous studies provide supporting observations: the effects of oxidative stress on dopaminergic neurones are known (3, 13) and the protection of dopaminergic neurones by melatonin has been described in many studies, for many different species (44).

It appears possible to discern trends for the normal peripheral plasma concentrations of serotonin, melatonin and dopamine in a healthy horse before it becomes aged then re-examine the individual on a bi-annual or quadti-annual basis to track any significant deviations from this baseline. Such an approach would mean that early veterinary intervention is possible to prevent the onset of the clinical signs under scrutiny. However, from a PPID perspective, we believe the ability to “re-balance” the neurohormones in an aged horse already exhibiting the clinical signs of PPID (where pre-existing data for neurohormone determinations does not exist) may be critical to its recovery. Our investigations leading to this study involved just such a horse. The individual had been scientifically diagnosed with PPID in 2001 and progressed, over the course of a two year period, to a state requiring euthanasia on the grounds of chronic lameness (subsequent to recurrent annual laminitis of up to Obel Grade 3 severity), low-grade pain and general ill health. Subsequent to our employing the methods of neurohormone rebalance described here, this horse now presents as clinically normal in appearance and to static scientific testing (ACTH, cortisol, insulin, glucose, etc.); it has returned to work as sound (see Example H, below),

We believe it is reasonable to suggest that both serotonin and dopamine must be in balance within an individual for that individual not to contract laminitis, but that change in dopamine concentration is, in fact, the key instigator of the pathogenesis; this may begin to explain why serotonin antagonists are an effective treatment in some horses, yet dopamine agonists appear to work well in others and are acknowledged as the more reliable regime. To put this in context of laminitis as a whole, we suggest that it is not changes in intestinal mucosal permeability per se, mediating trigger factors gaining access to the peripheral circulation (5), or necessarily the effect of neurohormonal or pre-cursor neurohormonal substances arising from trigger factors being themselves in the peripheral circulation (11, 39), but the effects of such on the neuroendocrine cascade of control, i.e. a more general systemic effect as opposed to direct or indirect effects via specific substances on the digital tissues and vessels associated with the hoof. Previously, basal plasma neurohormone concentrations were not considered as indices of system function since they are an expression of peripheral release. There are now several studies supporting the role of neurohormones or pre-cursor neurohormonal factors being implicated in the pathogenesis of laminitis from this viewpoint: the catecholamines, 5-HT and endothelin have been shown to represent some of the most potent vasoconstrictors of the equine digital circulation, digital arteries have been shown to be 30-40 fold more sensitive to vasoconstrictive actions of 5-HT than other peripheral blood vessels and vasoconstriction, possibly accompanied by an inflammatory stimulus, has been shown to lead to local platelet aggregation and disturbances in nutrient blood flow, causing injury to the lamellar epithelium (38). However, we believe that such substances, although no doubt contributory to laminitis in their effects, are effectors of, and mediated by a cascade of neuroendocrine control which begins with interaction between the pineal and pituitary glands and hypothalamus.

To summarise, previous studies have described PPID as a direct result of damage to specific parts of the central dopaminergic system and laminitis as the result of neurohormone or neurohormone precursor substances directly affecting the digit (with the possibility of additional indirect effects via central or peripheral systems). Our hypothesis is different in that it examines PPID, PPID associated laminitis and independently occurring laminitis in association with the concept of loss of central and peripheral neurohormone sensitivity over time, in the context of the Neuroendocrine Theory of Aging (21). We propose that, via the Accumulational Model (21), “aging” is causal in the degeneration of the periventricular hypophyseal dopaminergic neurons thought to result in PPID (3) and then plays a further role in the condition via continued loss of central and peripheral neurohormone sensitivity; which individuals will succumb to PPID, and when, being determined by the Ecological and Genetic Models (21).

### REFERENCES

1. Johnson P H. The equine metabolic syndrome—peripheral Cushing's syndrome. Vet Clin Equine 2002; 18: 271-293.
2. Schott H C. Pituitary pars intermedia dysfunction: equine Cushing's disease. Vet Clin Equine 2002; 18: 237-270.
3. McFarlane D, Donaldson M T, Saleh T M, Cribb A E. The Role of Dopaminergic Neurodegeneration in Equine Pituitary Pars Intermedia Dysfunction (Equine Cushing's Disease). In: The 49^{th} Annual Convention of the American Association of Equine Practitioners. New Orleans, La.: 2003. Available from Internet <http://www.ivis.org/proceedings/AAEP/2003/mcfarlane/chapter form.asp?L A=1> (17/03/2007).
4. Hood D M. Laminitis as a systemic disease. Vet Clin North America Equine Practice 1999; 15: 481-494.
5. Bailey S R, Marr C M, Elliott J. Current research and theories on the pathogenesis of acute laminitis in the horse. Vet J 2004; 167: 129-142.
6. French K R, Pollitt C C. Equine laminitis: glucose deprivation and MMP activation induce dermo-epidermal separation in vitro. Equine Vet J 2004 April; 36(3): 261-6.
7. Elliott J, Bailey S R. Gastrointestinal derived factors are potential triggers for the development of acute equine laminitis. J Nutr 2006 July; 136(7 Suppl): 21035-2107S.
8. Harris P. Bailey S R, Elliott J. Longland A. Countermeasures for pasture-associated laminitis in ponies and horses. J Nutr 2006 July; 136(7 Suppl): 2114S-2121S. Review.
9. Einckley K A & Henderson I W. The epidemiology of equine laminitis in the UK. Proceedings of the 35^{th} Congress of the British Equine Veterinary Congress, Warwick, 1996: p 62.
10. Bailey S R. The cost of laminitis: Implications for equine health and welfare and economic importance. Proceedings of Applying Equine Science: research into business, The Royal Agricultural College UK, 20-21 September 2005: p 15.
11. Elliott J, Berhane Y, Bailey S R. Effects of monoamines formed in the cecum of horses on equine digital blood vessels and platelets. AJVR 2003 Sept; 64(9): 1124-1131.
12. Bailey S R, Katz L M, Berhane Y, Samuels T, De Brauvere N, Marr C M Elliott J. Seasonal changes in plasma concentrations of cecum-derived amines in clinically normal ponies and ponies predisposed to laminitis. AJVR 2003 Sept; 64(9) 1132-1138.
13. McFarlane D, Dybdal N, Donaldson M T, Miller L, Cribb A E. Nitration and increased alpha-synuclein expression associated with dopaminergic neurodegeneration in equine pituitary pars intermedia dysfunctions. J Neuroendocrinol. 2005 February; 17(2): 73-80.
14. Piccione G, Assenza A, Fazio F, Percipalle M, Caola G. Central Fatigue and Nycthemeral Change of Serum Tryptophan and Serotonin in the Athletic Horse. J Circ Rhythms 2005; 3: 6. Short Paper,
15. Diekman M A, Braun W, Peter D, Cook D. Seasonal serum concentrations of melatonin in cycling and non-cycling mares. J. Anim. Sci. 2002; 80: 2949-2952.
16. Fitzgerald B P, McManus C J. Photoperiodic versus metabolic signals as determinants of seasonal anestrus in the mare. Biol. Reprod. 2000 July; 63(1): 335-40.
17. Reaven G M. Role of insulin resistance in human disease. Diabetes 1988; 37: 1595-1607.
18. Reaven G M, Strom T K, Fox B. Syndrome X, The Silent Killer: The New Heart Disease Risk. New York: Simon and Schuster, 2001.
19. Balasubramanyam A. Is the ‘metabolic syndrome’ a mild form of cushing's syndrome? The curious story of 11beta-hydroxysteroid dehydrogenase. In: Medscape Conference Coverage of ENDO 2002: The 84^{th} Annual Meeting of The Endocrine Society. San Francisco: 19-22 Jun. 2002. Available from Internet <http://www.medscape.com/viewarticle/440104> (22/04/2007).
20. Johnson P J, Slight S H, Ganjam V K, Kreeger J M. Glucocorticoids and Laminitis in the Horse. Vet Clin Equine 2002; 18: 219-236.
21. Dilman V M and Young J K. Development, Aging and Disease—A New Rationale for an Intervention Strategy. Switzerland: Harwood Academic Publishers, 1994.
22. Dilman V M, Revskoy S Y, Golubev A G. Neuroendocrine-ontogenetic mechanism of aging: toward an integrated theory of aging. Int Rev Neurobiol 1986; 28: 89-156.
23. Rozencwaig R, Grad B R and Ochoa J. The role of melatonin and serotonin in aging. Med. Hypothesis (England) 1987; 23(4): 337-352.
24. Rozencwaig R and Walji H. The Melatonin and Aging Sourcebook. Prescott: Hohm Press, 1997.
25. Humbert W and Pevet P. The Decrease of Pineal Melatonin Production with Age. Ann NY Acad Sci 1994; 719: 43-63.
26. Simpkins J W, Mueller G P, Huang H H, Meites J. Evidence for depressed catecholamine and enhanced serotonin metabolism in aging male rats: possible relation to gondotropin secretion. Endocrinology 1977; 100(6): 1672-1678.
27. Cano P, Cardinali D P, Jimenez V, Chacon F, Cutrera R A, Esquifino A I. Effect of Aging on 24-Hour Changes in Serotonin and Dopamine Turnover, and Somatostatin and Amino Acid Content, of the Rat Hypothalamus and Pituitary Gland. Biological Rhythm Research 2003; 34(3): 279-294.
28. Santanavanich C, Chetsawang B, Ebadi M, Govitrapong P. Effects of D1 and D2 dopamine receptor activation on melatonin synthesis in bovine pinealocytes, J. Pineal Res. 2003; 35(3): 169-176.
29. Abilio V C, Vera J A Jr, Ferreira L S, Duarte C R, Martins C R, Torres-Leite D, Ribeiro Rde A, Frussa-Filho R. Effects of melatonin on behavioural dopaminergic supersensitivity. Life Sci. 2003; 72(26): 3003-3015.

30. Smith A. Adult diabetes now destroying young lives. Available from Internet http://www.smb.com.au/articles/2004/03/16/1079199227720.html (22/04/2007)
31. Love S. Equine Cushing's Disease. Br Vet J 1993; 149(2): 139-153.
32. Keen J A, McLaren M, Chandler K J, McGorum BC. Biochemical indices of vascular function, glucose metabolism and oxidative stress in horses with equine Cushing's disease. Equine Vet J 2004 April; 36(3): 226-9.
33. Coffman J R, Colles C M. Insulin tolerance in laminitic ponies. Can J Comp Med 1983; 47(3): 347-351.
34. Jeffcott L B, Field J R. Current concepts of hyperlipaemia in horses and ponies. Vet Rec 1985; 116(17): 461-466.
35. Johnson P J, Ganjam V K, Slight S H, Kreeger J M, Messer N T. Tissue-specific dysregulation of cortisol metabolism in equine laminitis. Equine Vet. J. 2004; 36(1): 41-45.
36. Treiber K H, Kronfeld D S, Hess T M, Byrd B M, Splan R K and Staniar W B. Insulin resistance, inheritance and pre-laminitic metabolism syndrome in ponies. J Am Vet Med Assn 2006; accepted for publication.
37. Bailey S R, Elliott J. Plasma 5-hydroxytryptamine constricts equine digital blood vessels in vitro: implications for pathogenesis of acute laminitis. Equine Vet. J. 1998; 30(2):124-130.
38. Bailey S R, Elliott J. Evidence for different 5-HT_{1B/1D} receptors mediating vasoconstriction of equine digital arteries and veins. European Journal of Pharmacology; 335: 175-187.
39. Bailey S R, Cunningham F M, Elliott J. Endotoxin and dietary amines may increase plasma 5-hydroxytryptamine in the horse. Equine Vet. J. 2000; 32(6):497-504.
40. Podolak M, Kedzierski W, Bergero D. Comparison of the blood plasma catecholamines level in thoroughbred and Arabian horses during the same-intensity exercise. Pol. J. Vet. Sci. 2006; 9(1): 71-3.
41. Guillaume D, Zarazaga L A, Malpaux B, Chemineau P. Variability of plasma melatonin level in pony mares (Equus caballus), comparison with the hybrid: mules and with jennies (Equus asinus). Reprod Nutr Dev. 2006 November-December; 46(6): 633-9.
42. Amenta, F (ed.). Peripheral Dopamine Pathophysiology. Florida, USA: CRC Press Inc., 2000.
43. Bailey S R, Berhane Y, Marr C M, Elliott J. Effects of vasoactive amines from the equine hindgut on digital blood flow in the normal horse. Journal of Veterinary Internal Medicine 2002; 16: 335.
44. Singh S, Ahmed R, Sagar R K, Krishana B. Neuroprotection of the nigrostriatal dopaminergic neurons by melatonin in herniparkinsonium rat. Indian J. Med. Res. 2006; 124: 419-426.

### Example F

### Statistical Analysis of Data I

### Stage 1

A three-way split plot ANOVA was carried out, with the following fitting effects:
Month¹
Condition¹
Horse¹
Month*Condition¹
Horse*Month*Condition (Main Plot error)
Time
Time*Month
Condition*Month,
Condition*Time*Month

The terms marked¹ were tested against the main plot error, the remaining terms against the split plot error.

This analysis was used to identify outliers and to cheek the distribution of the residual variability. This was assessed graphically as reasonably normally distributed but showed some extreme outliers. Our assessment is that Analysis of Variance is a valid method of analysis of the data.

From this analysis the data was screened for outliers (19 were identified), 3 of which were removed from further analysis.

### Stage 2

The three-way ANOVA was re-run omitting the outliers and the following effects detected as significant.

TABLE F1
F Test p-values for each condition.

| Effect | Melatonin | Serotonin | Dopamine | Serotonin:Melatonin | Dopamine:Melatonin | Dopamine:Seratoninn |
|---|---|---|---|---|---|---|
| Month | 0.0015 | NS | 0.0554 | 0.0327 | 0.014 | NS |
| Condition | NS | 0.0783 | 0.0014 | 0.0007 | 0.0046 | NS |
| Condition * month | NS | NS | 0.0996 | NS | NS | NS |
| Horse | <0.0001 | 0.002 | 0.0014 | 0.01 | NS | NS |
| Time | <0.0001 | 0.016 | 0.0009 | <0.0001 | <0.0001 | NS |
| Time * condition | NS | 0.0649 | NS | 0.1063 | 0.0464 | NS |
| Time * Month | 0.0203 | 0.0188 | NS | <0.0001 | <0.0001 | NS |
| Time * condition * month | NS | NS | 0.0778 | <0.0001 | <0.0001 | NS |

p-values are given for significance levels below 0.15 - otherwise marked NS (Not Significant)

Investigation of the monthly data revealed month on month variation in the horses tested in the trial and the number of tests carried out on each horse within each month. As a consequence, a further analysis was performed in which the condition effects were assessed through analysis of variance of the data separately for each month.

### Stage 3

Analysis of Variance results (by Month)

Analysis of Variance models were fitted by month, with the following fitting effects:
Condition
Horses within condition (MP Error)
Time
Condition*Time

Table F2 below gives significance of the F Tests for effects from Split Plot Analysis of variance. Condition tested against Main plot error (horses within condition). Time & Time by condition tested against split plot error.

TABLE F2
F Test p-values for each condition (month by month analysis)

| Month | Effect | Melatonin | Serotonin | Dopamine | Serotonin:Melatonin | Dopamine:Melatonin | Dopamine:Serotonin |
|---|---|---|---|---|---|---|---|
| June 05 | Condition | NS | NS | 0.0714 | 0.0855 | 0.0409 | NS |
| | Time | <0.0001 | 0.0452 | NS | 0.0036 | 0.0106 | NS |
| | Time * condition | NS | NS | 0.0773 | NS | NS | 0.1204 |
| September | Condition | NS | NS | 0.0733 | NS | NS | NS |
| 2005 | Time | <0.0001 | 0.1496 | 0.0629 | 0.0294 | 0.0252 | NS |
| | Time * condition | NS | NS | 0.4137 | 0.1288 | NS | NS |
| December | Condition | NS | NS | NS | 0.0551 | 0.0986 | NS |
| 2005 | Time | <0.0001 | 0.0074 | NS | <0.0001 | <0.0001 | NS |
| | Time * condition | NS | NS | NS | <0.0001 | 0.0003 | NS |
| March | Condition | NS | NS | NS | NS | NS | NS |
| 2006 | Time | <0.0001 | NS | <0.0001 | <0.0001 | <0.0001 | NS |
| | Time * condition | NS | NS | 0.0456 | NS | NS | NS |

p-values are given for significance levels below 0.15 - otherwise marked NS (Not Significant)

### Comparison of Controls

The difference in adjusted mean scores (LSMEANS) between Time 1 (13.00 start day) and Time 9 (13.00 end day) was tested for each “treatment” group using a T-test (see Table F3).

TABLE F3
F Test p-values for each condition (month by month analysis)

| | | Significance level t Test | | |
|---|---|---|---|---|
| | | Comparing Mean Start day with mean End day | | |
| | | Melatonin | Serotonin | Dopamine |
| June 2005 | C | NS | 0.0449 | NS |
| | N | NS | NS | 0.0809 |
| September 2005 | C | NS | NS | NS |
| | N | NS | NS | NS |
| December 2005 | C | NS | NS | NS |
| | N | NS | NS | NS |
| March 2006 | C | | NS | NS |
| | N | | NS | 0.0045 |

Apart from the Dopamine result for N (Normal horses), the number of significant results are no more than could be expected by chance indicating that overall there is no strong evidence of a difference between the average hormone levels on the two control days.

### Comparison of “Treatments” by Time

Pairwise comparisons (using T-Tests) of the differences in adjusted means at each time point is given in the Table F4 to F7 below.

TABLE F4
Pairwise comparisons (using T-Tests) for June

| | Significant level comparison of treatments at | | | | | |
|---|---|---|---|---|---|---|
| Time | each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | NS | 0.046 | 0.0452 | NS |
| 2-16.00 | NS | NS | NS | NS | NS | NS |
| 3-19.00 | NS | 0.0528 | 0.0095 | NS | 0.0336 | 0.0552 |
| 4-22.00 | NS | NS | 0.147 | NS | NS | 0.0461 |
| 5-01.00 | 0.026 | 0.1206 | NS | NS | NS | NS |
| 6-04.00 | NS | NS | NS | NS | NS | NS |
| 7-07.00 | NS | 0.0224 | 0.1433 | 0.0026 | 0.0807 | NS |
| 8-10.00 | NS | NS | NS | NS | NS | NS |
| 9-13.00 | NS | NS | 0.003 | 0.0325 | 0.0027 | 0.0549 |

p-values are given for significance levels below 0.15 - otherwise marked NS (Not Significant)

TABLE F5
Pairwise comparisons (using T-Tests) for September

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | 0.0868 | NS | NS | NS |
| 2-16.00 | NS | NS | 0.0328 | 0.0007 | 0.006 | NS |
| 3-19.00 | NS | NS | NS | NS | NS | NS |
| 4-22.00 | NS | NS | 0.0004 | NS | NS | NS |
| 5-01.00 | NS | NS | 0.0637 | NS | NS | 0.0126 |
| 6-04.00 | NS | NS | NS | NS | NS | NS |
| 7-07.00 | NS | NS | NS | NS | NS | NS |
| 8-10.00 | NS | 0.0654 | NS | NS | NS | NS |
| 9-13.00 | NS | NS | 0.073 | NS | 0.0826 | NS |

p-values are given for significance levels below 0.15 - otherwise marked NS (Not Significant)

TABLE F6
Pairwise comparisons (using T-Tests) for December

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | 0.0155 | NS | NS | NS | NS |
| 2-16.00 | NS | NS | NS | NS | NS | NS |
| 3-19.00 | NS | NS | NS | NS | NS | NS |
| 4-22.00 | NS | NS | NS | NS | NS | 0.0739 |
| 5-01.00 | NS | 0.0152 | NS | NS | NS | 0.1344 |
| 6-04.00 | 0.0439 | NS | NS | NS | NS | NS |
| 7-07.00 | 0.0698 | NS | NS | NS | NS | NS |
| 8-10.00 | NS | NS | NS | <0.0001 | <0.0001 | NS |
| 9-13.00 | NS | NS | NS | NS | NS | NS |

p-values are given for significance levels below 0.15 - otherwise marked NS (Not Significant)

TABLE F7
Pairwise comparisons (using T-Tests) for March

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | 0.0543 | 0.038 | 0.0342 | NS |
| 2-16.00 | NS | NS | NS | NS | NS | NS |
| 3-19.00 | NS | NS | 0.1123 | NS | NS | 0.0486 |
| 4-22.00 | NS | 0.0889 | 0.0158 | NS | 0.0529 | NS |
| 5-01.00 | NS | NS | 0.0722 | NS | NS | NS |
| 6-04.00 | 0.0986 | NS | NS | NS | NS | NS |
| 7-07.00 | NS | NS | 0.1322 | NS | NS | NS |
| 8-10.00 | NS | NS | NS | 0.01 | 0.0633 | NS |
| 9-13.00 | | NS | NS | | | NS |

p-values are given for significance levels below 0.15 - otherwise marked NS (Not Significant)

### Example G

### Statistical Analysis of Data II

The original analysis described in Example F above was carried out based on a single analysis of variance within each season for each hormone measure (direct and ratio). To allow for diurnal variation in magnitude of some hormones, an alternative analysis was performed split by daylight (dark and light) within each season.

The 3 outliers identified in the original analysis were removed from the data. The comparisons were again carried out using LSMEANS to adjust for the imbalance in the monthly data due to horses whose test results are not reported.

### Phase 2 Analysis

Analysis of Variance results (by Month and Daylight Dark/Light)

Analysis of Variance models were fitted by month and daylight, for the following fitting effects:
Condition
Horses within condition (MP Error)
Time (within daylight period)
Condition*Time (within daylight period)

The ratios of the hormones were also analysed.

Examination of the residuals indicated that, within the two daylight periods defined, the assumption of constant horse to horse variation in hormone measures was better satisfied in this analysis split by daylight than in the original global analysis.

The tables below give significance of the F Tests for effects from Split Plot Analysis of variance. Condition tested against Main plot error (horses within condition). Time & Time by Condition tested against split plot error.

p-values are given for significance levels below 0.15—otherwise marked NS (Not Significant)

TABLE G1
Dark Time Analysis

| Month | Effect | Melatonin | Serotonin | Dopamine | Serotonin:Melatonin | Dopamine:Melatonin | Dopamine:Serotonin |
|---|---|---|---|---|---|---|---|
| June 05 | Condition | NS | NS | NS | NS | NS | NS |
| | Time | NS | 0.0371 | NS | 0.1265 | NS | NS |
| | Time * condition | NS | NS | 0.0416 | NS | 0.0760 | NS |
| September | Condition | NS | NS | 0.0666 | NS | 0.1382 | NS |
| 2005 | Time | NS | NS | NS | NS | NS | NS |
| | Time * condition | NS | NS | NS | NS | 0.0818 | NS |
| December | Condition | NS | NS | NS | NS | NS | NS |
| 2005 | Time | 0.1031 | 0.0222 | 0.0802 | NS | 0.1174 | NS |
| | Time * condition | NS | NS | NS | NS | NS | NS |
| March | Condition | NS | NS | NS | NS | NS | NS |
| 2006 | Time | 0.1062 | NS | <0.0001 | NS | 0.1093 | NS |
| | Time * condition | NS | NS | 0.0123 | NS | NS | NS |

TABLE G2
Light Time Analysis

| Month | Effect | Melatonin | Serotonin | Dopamine | Serotonin:Melatonin | Dopamine:Melatonin | Dopamine:Serotonin |
|---|---|---|---|---|---|---|---|
| June 05 | Condition | 0.0524 | NS | 0.0277 | 0.0883 | 0.0302 | NS |
| | Time | NS | 0.1361 | NS | NS | NS | 0.0815 |
| | Time * condition | NS | NS | NS | NS | NS | NS |
| September | Condition | NS | NS | 0.0914 | NS | NS | NS |
| 2005 | Time | 0.0287 | NS | 0.0813 | NS | NS | NS |
| | Time * condition | NS | NS | NS | NS | NS | NS |
| December | Condition | 0.1335 | NS | NS | 0.0356 | 0.0643 | NS |
| 2005 | Time | NS | 0.0317 | NS | 0.0075 | 0.0391 | NS |
| | Time * condition | NS | 0.1009 | NS | <0.0056 | 0.028 | NS |
| March | Condition | NS | NS | NS | NS | NS | NS |
| 2006 | Time | 0.0541 | NS | 0.0176 | NS | NS | NS |
| | Time * condition | NS | NS | NS | NS | NS | NS |

### Comparison of Controls

The difference in adjusted mean scores (LSMEANS) between Time 1 (13.00 start day) and Time 9 (13.00 end day) was tested for each “treatment” group using a T-test.

TABLE G3
Comparison of controls

| | | Significance level t Test | | |
|---|---|---|---|---|
| | | Comparing Mean Start day with mean End day | | |
| | | Melatonin | Serotonin | Dopamine |
| June 2005 | C | NS | 0.0370 | NS |
| | N | NS | NS | 0.0756 |
| September 2005 | C | NS | NS | NS |
| | N | NS | NS | NS |
| December 2005 | C | NS | NS | NS |
| | N | NS | NS | NS |
| March 2006 | C | | NS | NS |
| | N | | NS | 0.011 |

Apart from the Dopamine result for N (Normal horses) the number of significant results is no more than could be expected by chance, indicating that overall there is no strong evidence of a difference between the average hormone levels on the two control days.

### Comparison of “Treatments” by Time

Pairwise comparisons (using T Tests) of the differences in adjusted means at each time point (analysed by daylight) are given in Tables G4 to G7 below.

TABLE G4
Pairwise comparisons (using T-Tests) for June

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | NS | 0.0836 | 0.1016 | NS |
| 2-16.00 | 0.0017 | NS | NS | NS | NS | NS |
| 3-19.00 | 0.0735 | 0.0439 | 0.0192 | NS | 0.0449 | 0.0633 |
| 4-22.00 | NS | NS | 0.0243 | 0.1342 | 0.0816 | 0.056 |
| 5-01.00 | NS | 0.0318 | NS | 0.0774 | NS | NS |
| 6-04.00 | NS | NS | NS | 0.0056 | 0.1232 | NS |
| 7-07.00 | 0.0102 | 0.0094 | 0.1482 | 0.0112 | 0.0893 | NS |
| 8-10.00 | 0.0202 | NS | NS | NS | NS | NS |

p-values are given for significance levels below 0.15 - otherwise marked NS (Not Significant)

TABLE G5
Pairwise comparisons (using T-Tests) for September

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | 0.1016 | NS | NS | NS |
| 2-16.00 | NS | NS | 0.0419 | 0.0067 | 0.0254 | NS |
| 3-19.00 | 0.0469 | NS | NS | NS | NS | NS |
| 4-22.00 | NS | NS | 0.0019 | 0.0195 | 0.0005 | NS |
| 5-01.00 | NS | NS | 0.0825 | 0.037 | NS | 0.1216 |
| 6-04.00 | NS | NS | NS | NS | NS | NS |
| 7-07.00 | NS | NS | NS | NS | NS | NS |
| 8-10.00 | NS | 0.0833 | NS | NS | NS | NS |

p-values are given for significance levels below 0.15 - otherwise marked NS (Not Significant)

TABLE G6
Pairwise comparisons (using T-Tests) for December

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | 0.003 | NS | NS | NS | NS |
| 2-16.00 | 0.0632 | NS | NS | NS | NS | NS |
| 3-19.00 | NS | NS | NS | NS | NS | NS |
| 4-22.00 | NS | NS | NS | 0.0164 | 0.117 | NS |
| 5-01.00 | NS | 0.0335 | NS | <.0001 | NS | NS |
| 6-04.00 | 0.0977 | NS | NS | 0.0426 | NS | NS |
| 7-07.00 | 0.1412 | NS | NS | 0.0327 | 0.0052 | NS |
| 8-10.00 | NS | NS | NS | <0.0001 | 0.0014 | NS |

p-values are given for significance levels below 0.15 - otherwise marked NS (Not Significant)

TABLE G7
Pairwise comparisons (using T-Tests) for March

| | Significant level comparison of treatments | | | | | |
|---|---|---|---|---|---|---|
| Time | at each time point using adjusted (LSMEANS) | | | | | |
| period | Melatonin | Serotonin | Dopamine | Serotonin:Melatonin | Dopamine:Melatonin | Dopamine:Serotonin |
| 1-13.00 | NS | NS | 0.0797 | NS | 0.0985 | NS |
| 2-16.00 | NS | NS | NS | NS | NS | NS |
| 3-19.00 | NS | NS | 0.0825 | 0.0438 | NS | NS |
| 4-22.00 | NS | 0.1133 | 0.0097 | 0.0335 | 0.0112 | NS |
| 5-01.00 | NS | NS | 0.0503 | 0.0037 | NS | NS |
| 6-04.00 | 0.072 | NS | NS | NS | NS | NS |
| 7-07.00 | NS | NS | NS | NS | NS | NS |
| 8-10.00 | 0.0853 | NS | NS | 0.0638 | 0.0983 | NS |

p-values are given for significance levels below 0.15 - otherwise marked NS (Not Significant)

### Example H

### Evidence of Treatment Efficacy

The objectives of the study were to discover whether a pony that had been scientifically diagnosed with pituitary-dependent Cushing's syndrome for several years (and was due to be euthanised) might be returned to an acceptable quality of life within six months, then subsequently to gentle work, It was proposed that instead of treating chronic laminitis in the individual in terms of what was occurring in the hoof, the laminitis should be treated as stemming from a distinct neuroendocrine imbalance; the key to stopping the laminitis was in correcting this imbalance. A treatment regime to do this was carefully designed and administered (see below).

When a horse experiences an episode of laminitis, the soft tissue attachment between the third phalanx (P3 or Pedal bone) and the interior of the hoof capsule is degraded or weakened. Due to this, plus the weight of the horse together with the pull of the Deep Digital Flexor Tendon (which attaches at the back of the P3 and continues up the leg), the third phalanx and whole bone column may move downward towards the sole (but remain almost parallel to the ground—‘sinking’) or the toe of the third phalanx itself may appear to move towards the sole (‘rotation’). In the scan shown in FIG. 10(A), this ‘rotation’ can be seen to have taken place; the front edge of the P3 should be parallel to the exterior hoof wall, and the underside of the P3 almost parallel with the ground surface. The black markings show a veterinary prescription for corrective trimming to try to address this misalignment. At its most extreme, ‘rotation’ may involve the tip of the P3 emerging through the underside of the sole.

The horse was treated in accordance with the proposed theory of neurotransmitter balance. Specifically, the horse received the following agents on a daily basis:

(a) Treatment regime during acute laminitis

| Time of | | |
|---|---|---|
| administration | Agent (dose) | Activity |
| 19:00 | Pergolide (0.75 mg) | Dopamine agonist |
| 09:00 & 19:00 | Feverfew* (800 mg/dose) | Serotonin antagonist |
| 00:00 | Melatonin (1.5 mg) | Melatonin agonist |

*Perthenolide at 0.2% standardisation

(b) Maintenance regime after recovery from acute laminitis

| Time of | | |
|---|---|---|
| administration | Agent (dose) | Activity |
| 19:00 to 22.00 | Pergolide (0.75 mg) | Dopamine agonist |
| 09:00 & 19:00 | Feverfew* (800 mg/dose) | Serotonin antagonist |
| 19:00 to 22.00 | Melatonin (1.5 mg) | Melatonin agonist |

*Perthenolide at 0.2% standardisation

Unfortunately, as in most of these cases, the only way to help the animal was to let the damaged hoof grow out whilst applying corrective farriery to the affected hooves; it may take over nine months for a completely new hoof to have grown in place of the old one. The problem with Cushingoid animals is that their body state means that there is little new hoof growth. Thus, each successive episode of laminitis means that damage accumulates within the affected hooves, often to the point at which the horse must be euthanised because of the chronic pain it is in. Fortunately, in this case, the treatment meant that new hoof growth was forthcoming, and, about nine months later, it can be seen that both the angle between the front edge of the P3 and the hoof wall, and the underside of the P3 and the ground have improved substantially (see FIG. 10(B)).

### CONCLUSIONS

The study was successful in its objective and the pony is now 32 years old and has been returned to ridden work. In summary, abnormal biochemistries and blood analyses were returned to within normal range, hormonal determinations, such as ACTH cortisol and insulin, were also returned to within normal range and external clinical signs of the condition were no longer present. This is in contrast to many advanced cases of equine Cushing's syndrome in which a dopamine agonist or serotonin antagonist is administered; the pony appeared to have been returned to a pre-Cushingoid state

1. A method for diagnosing a disorder or disease associated with an imbalance of monoamines in a subject, or of diagnosing susceptibility of the subject to the same, wherein the method comprises the following steps:
(a) providing a sample from a subject to be tested;
(b) measuring the levels of dopamine, melatonin and/or serotonin in the sample; and
(c) comparing the levels measured in step (b) with levels of dopamine, melatonin and/or serotonin associated with a reference population of subjects
wherein a difference in one or more of the levels measured in step (b) with the levels of dopamine, melatonin and serotonin associated with the reference population of subjects is indicative of a positive diagnosis.

2. A method according to claim 1 wherein the subject to be tested is selected from the group consisting of human, equine and canine subjects.

3. A method according to claim 1 wherein the subject to be tested is equine.

4. A method according claim 1 wherein the disorder or disease associated with an imbalance of monoamines is a disorder, disease or condition associated with aging.

5. A method according to claim 3 wherein the disorder or disease associated with an imbalance of monoamines is laminitis.

6. A method according to claim 3 wherein the disorder or disease associated with an imbalance of monoamines is equine Cushing's syndrome.

7. A method according to claim 3 wherein the disorder or disease associated with an imbalance of monoamines is equine metabolic syndrome (Reaven's syndrome).

8. A method according to claim 7 wherein the sample is a blood plasma sample.

9. A method according to claim 8 wherein the blood plasma sample is treated with EDTA.

10. A method according to claim 1 wherein the levels of dopamine, melatonin and/or serotonin are measured by radioimmunoassay.

11. A method according to claim 1 wherein the reference population of subjects comprise healthy, matched subjects.

12. A method according to claim 1 wherein the levels of dopamine, melatonin and/or serotonin associated with predetermined population of subjects are matched for time of sample collection.

13. A method according to claim 12 wherein step (b) further comprises measuring one or more of glucose, ACTH, cortisol and insulin and step (c) further comprises comparing the levels measured in step (b) with levels of glucose, ACTH, cortisol and/or insulin associated with a reference population of subjects.

14. A method for treating a subject suffering from a disorder or disease associated with an imbalance of monoamines, or from a susceptibility to the same, the method comprising the following steps:
(a) identifying an imbalance in one or more of the levels of dopamine, melatonin and/or serotonin in a sample from the subject compared to levels of dopamine, melatonin and/or serotonin associated with reference subjects by:
i. providing a sample from a subject to be tested;
ii. measuring the levels of dopamine, melatonin and/or serotonin in the sample;
iii. comparing the levels measured in step (b) with levels of dop amine, melatonin and/or serotonin associated with a reference population of subjects
wherein a difference in one or more of the levels measured in step (b) with the levels of dopamine, melatonin and serotonin associated with the reference population of subjects is indicative of a positive diagnosis; and
(b) administering one or more agent(s) to try to restore the levels of dopamine, melatonin and/or serotonin in the subject to levels of dopamine, melatonin and/or serotonin associated with reference subjects.

15. A method according to claim 14 wherein the subject to be tested is selected from the group consisting of human, equine and canine subjects.

16. A method according to claim 14 wherein the subject to be tested is equine.

17. A method according to claim 16 wherein the disorder or disease associated with an imbalance of monoamines is a disorder, disease or condition associated with aging.

18. A method according to claim 16 wherein the disorder or disease associated with an imbalance of monoamines is laminitis.

19. A method according to claim 16 wherein the disorder or disease associated with an imbalance of monoamines is equine Cushing's syndrome.

20. A method according to claim 16 wherein the disorder or disease associated with an imbalance of monoamines is equine metabolic syndrome (Reaven's syndrome).

21. A method according to claim 14 wherein the one or more agents administered in step (b) are selected from the group consisting of dopamine agonists, dopamine antagonists, serotonin agonists, serotonin antagonists, melatonin agonists, melatonin antagonists.

22. A method according to claim 14 wherein the one or more agents administered in step (b) includes a dopamine agonist

23. A method according to claim 18 wherein the dopamine agonist is selected from the group consisting of pergolide, cabergoline, ropinirole, bupropion, apomorphine, L-DOPA, dopamine, bromocriptine, lisuride, selegiline, St John's Wort, pramipexole, amantadine, chasteberry (Agnus castus), blueberries and other dark fruits.

24. A method according to claim 22 wherein the dopamine agonist is pergolide.

25. A method according to claim 21 wherein the one or more agents administered in step (b) includes a serotonin antagonist.

26. A method according to claim 25 wherein the serotonin antagonist is selected from the group consisting of feverfew, cyproheptadine, methysergide, isocarboxazid, phenelzine, selegiline and tranylcypromine.

27. A method according to claim 25 wherein the serotonin antagonist is feverfew or cyproheptadine.

28. A method according to claim 14 wherein the one or more agents administered in step (b) includes a melatonin agonist.

29. A method according to claim 28 wherein the melatonin agonist is melatonin.

30. A method according to claim 14 wherein step (b) comprises administering a dopamine agonist, a serotonin antagonist and/or melatonin.

31. A method according claim 14 wherein step (b) comprises or consists of administering a dopamine agonist and a serotonin antagonist.

32. A method according to claim 14 wherein step (b) comprises administering pergolide, feverfew and melatonin.

33. A method according to claim 14 wherein in step (b) the one or more agents are administered at a time of day selected so as to mimic normal circadian rhythm.

34. A diagnostic kit for performing a method according to claim 1 comprising:
(a) one or more reagent(s) for the detection of dopamine;
(b) one or more reagent(s) for the detection of serotonin; and/or
(c) one or more reagent(s) for the detection of melatonin.

35. A diagnostic kit according to claim 34 comprising
(a) one or more reagent(s) for the detection of dopamine by radioimmunoassay;
(b) one or more reagent(s) for the detection of serotonin by radioimmunoassay; and/or
(c) one or more reagent(s) for the detection of melatonin by radioimmunoassay.

36. A diagnostic kit according to claim 34 further comprising instructions for performing a method according to claim 1.

37. A treatment kit for use in a method according to claim 14 comprising:
(a) one or more agent(s) for modulating the level of dopamine in a patient;
(b) one or more agent(s) for modulating the level of serotonin in a patient; and/or
(c) one or more agent(s) for modulating the level of melatonin in a patient.

38. A treatment kit according to claim 37 comprising:
(a) a dopamine agonist
(b) a serotonin antagonist; and/or
(c) a melatonin agonist.

39. A treatment kit according to claim 38 comprising or consisting of a dopamine agonist and a serotonin antagonist.

40. A treatment kit according to claim 39 comprising pergolide, feverfew and/or melatonin.

41. A treatment kit according to claim 38 further comprising instructions for performing a method according to claim 14.

42. An animal feed, or a supplement therefor, comprising:
(a) one or more agent(s) for modulating the level of dopamine in an animal;
(b) one or more agent(s) for modulating the level of serotonin in an animal; and/or
(c) one or more agent(s) for modulating the level of melatonin in an animal.

43. An animal feed, or supplement therefor, according to claim 42 comprising or consisting of a dopamine agonist and a serotonin antagonist.

44. An animal feed, or supplement therefor, according to claim 42 comprising precursors for dopamine, serotonin and/or melatonin.

45. An animal feed, or supplement therefor, according to claim 42 comprising tyrosine and parthenolide.

46. An animal feed, or supplement therefor, according to claim 40 further comprising melatonin.

47. A pharmaceutical composition for use in horses comprising a dopamine agonist, a serotonin antagonist, and a melatonin agonist.

48. A pharmaceutical composition according to claim 47 comprising melatonin (optionally in controlled-release form; e.g. 1.5 mg), pergolide (such as the mesilate; e.g. 0.75 mg) and parthenolide (e.g. 3.2 mg).

49-53. (canceled)
